(19) United States
(12) Patent Application Publication
(10) Pub. No.: US 2007/0021819 A1
(43) Pub. Date: Jan. 25, 2007

(54) Apparatus and Methods for Locating an Ostium of a Vessel

(76) Inventors: Jeff Krolik, Campbell, CA (US); Elliot Kim, Santa Clara, CA (US); James H. Dreher, Santa Monica, CA (US)

Correspondence Address: Vista IP Law Group LLP, 2040 MAIN STREET, 9TH FLOOR, IRVINE, CA, 92614, US

(21) Appl. No.: 11/419,997

(22) Filed: May 23, 2006

Related U.S. Application Data
(60) Provisional application No. 60/683,931, filed on May 23, 2005.

(51) Int. Cl. A61F 2/06

(52) U.S. Cl. 623/1.11

(57) ABSTRACT

Apparatus and methods are provided for locating an ostium of a body lumen. In one embodiment, a delivery catheter or other tubular member includes a distal end sized for introduction into a body lumen, and one or more locator loops on the distal end. In one embodiment, the locator loop may include first and second ends fixed to the distal end, first and second resilient struts extending from the first and second ends, respectively, and a curved intermediate region extending between the first and second struts. The loop may be resiliently compressible to a contracted condition for delivery and resiliently expandable to an enlarged condition when deployed. The apparatus may include one or more balloons, stents, and the like on the distal end adjacent the locator loop.

This application claims benefit of provisional application Ser. No. 60/683,931, filed May 23, 2005, the entire disclosure of which is expressly incorporated by reference herein.

### FIELD OF THE INVENTION

The present invention relates generally to apparatus and methods for locating an ostium of a blood vessel or other body lumen, and, more particularly, to apparatus and methods for locating an ostium of a blood vessel or other body lumen to deliver a stent or other prosthesis into or adjacent the ostium.

### BACKGROUND

Tubular endoprosthesis or “stents” have been suggested for dilating or otherwise treating stenoses, occlusions, and/or other lesions within a patient's vasculature or other body lumens. For example, a self-expanding stent may be maintained on a catheter in a contracted condition, e.g., by an overlying sheath or other constraint, and delivered into a target location, e.g., a stenosis within a blood vessel or other body lumen. When the stent is positioned at the target location, the constraint may be removed, whereupon the stent may automatically expand to dilate or otherwise line the vessel at the target location. Alternatively, a balloon-expandable stent may be carried on a catheter, e.g., crimped or otherwise secured over a balloon, in a contracted condition. When the stent is positioned at the target location, the balloon may be inflated to expand the stent and dilate the vessel.

Sometimes, a stenosis or other lesion may occur at an ostium or bifurcation, i.e., where a branch vessel extends from a main vessel. For example, such a lesion may form within a coronary artery immediately adjacent the aortic root. U.S. Pat. No. 5,749,890 to Shaknovich discloses a stent delivery assembly for placing a stent in an ostial lesion. U.S. Pat. No. 5,632,762 to Myler discloses a tapered balloon on a catheter for positioning a stent within an ostium. U.S. Pat. No. 5,607,444 to Lam discloses an expandable ostial stent including a tubular body and a deformable flaring portion. Published application US 2002/0077691 to Nachtigall discloses a delivery system that includes a sheath for holding a stent in a compressed state during delivery and a retainer that holds a deployable stop in an undeployed position while the delivery system is advanced to a desired location.

Accordingly, apparatus and methods for locating an ostium and/or for delivering a stent within an ostium would be useful.

### SUMMARY OF THE INVENTION

The present invention is directed to apparatus and methods for locating a branch body lumen extending from a main body lumen, and, more particularly, to apparatus and methods for locating an ostium or bifurcation of a blood vessel or other body lumen, e.g., for delivering a stent or other prosthesis within or adjacent the ostium and/or for accessing the blood vessel.

In accordance with one embodiment, an apparatus is provided that includes a tubular member including proximal and distal ends, and a lumen extending between the proximal and distal ends, an elongate member including a distal portion disposed within the lumen such that the distal portion may be advanced beyond the tubular member distal end, an expandable locator on the distal portion. In one embodiment, the locator includes a loop including first and second ends fixed to the distal portion, first and second resilient struts extending from the first and second ends, respectively, and a curved intermediate region extending between the first and second struts. The loop may be resiliently compressible to a contracted condition when the distal portion is disposed within the lumen and resiliently expandable to an enlarged condition when the distal portion is advanced beyond the tubular member distal end.

In an exemplary embodiment, the loop may substantially define a plane in the enlarged condition and/or the plane may define an acute angle with a longitudinal axis of the tubular member. In another embodiment, the loop may be twisted asymmetrically relative to a longitudinal axis of the tubular member in the enlarged condition.

In alternative embodiments, only a single loop or a plurality of expandable loops may be provided on the distal portion of the tubular member. If a plurality of expandable loops are provided, the loops may be disposed symmetrically or asymmetrically around a circumference of the tubular member. In one embodiment, the plurality of expandable loops may include curved intermediate regions that generally define a portion of an ellipse surrounding the distal portion.

In accordance with another embodiment, an apparatus is provided for locating an ostium of a body lumen. Generally, the apparatus includes an elongate member including a distal portion that may advanced through a guide catheter or other tubular member, and a plurality of expandable loops on the distal portion. Each loop may include first and second resilient struts extending from the distal portion, and a curved intermediate region extending between the first and second struts. In addition or alternatively, each loop may be resiliently compressible to a contracted condition when the distal portion is disposed within the tubular member and resiliently expandable to an enlarged condition when the distal portion is advanced from the tubular member.

In one embodiment, the loops may be disposed around the distal portion such that the intermediate regions define at least a portion of an ellipse surrounding the distal portion when the loops are in the enlarged condition. In addition or alternatively, the struts may be resiliently deflectable when the loops are expanded to the enlarged condition to provide tactile feedback when one or more of the intermediate regions contact an ostium.

Optionally, a tubular prosthesis may be provided on the distal portion, e.g., adjacent the loops such that the apparatus may be used to position the prosthesis within an ostium.

In accordance with still another embodiment, an apparatus is provided for locating an ostium of a body lumen that includes a tubular member including a proximal end, a distal end sized for introduction into a body lumen, a lumen extending between the proximal and distal ends, and a distal portion. One or more locator elements may be disposed asymmetrically on the distal portion, each locator element including a first end fixed to the distal portion and a second end free from the distal portion. Each locator element may be resiliently compressible to a contracted condition when the distal portion is disposed within a lumen of a delivery device, and each locator element being resiliently expandable to an enlarged condition when fully deployed from the delivery device.

Optionally, a stent or other prosthesis may be disposed on the distal portion.

In accordance with yet another embodiment, an apparatus is provided for locating an ostium of a body lumen that includes a tubular member including a proximal end, a distal end sized for introduction into a body lumen, a lumen extending between the proximal and distal ends, and a distal portion, and a locator loop on the distal portion. The locator loop may be resiliently compressible to a contracted condition when the distal portion is disposed within a lumen of a delivery device and/or resiliently expandable to an enlarged condition when fully deployed from the delivery device.

In one embodiment, the locator loop may include a loop that substantially surrounds the distal portion of the tubular member in the enlarged condition, and a plurality of struts extending between the loop and the distal portion for attaching the locator loop to the tubular member. Optionally, the struts may include an inner portion closer to the tubular member and an outer portion closer to the loop, the inner portion being more rigid than the outer portion. In addition or alternatively, one or more supports may extend between adjacent struts at intermediate regions of the struts.

In another embodiment, the locator loop may include a base attached to the distal portion of the tubular member, the struts extending from the base. Optionally, at least the base and the struts may be formed from a unitary tubular body. Optionally, the loop may also be formed from the unitary tubular body, or the loop may be formed from one or more wires attached to the unitary tubular body.

In accordance with still another embodiment, a method is provided for delivering a stent within an ostium communicating from a main body lumen to a branch body lumen. A distal end of a delivery catheter may be advanced into the main body lumen, the distal end including one or more locator elements constrained in a contracted condition. The one or more locator elements may be released within the main body lumen and directed against a wall of the ostium, and a procedure may be performed at or within the ostium based upon the position of the one or more locator elements in the enlarged condition, e.g., a stent may be delivered into the ostium.

In one embodiment, the one or more locator elements may assume an asymmetrical orientation upon being released and/or may cause the distal end of the delivery catheter to rotate about its longitudinal axis when the locator elements are released.

In addition or alternatively, the one or more locator elements may provide tactile feedback resisting further advancement when the one or more locator elements contact the main body lumen wall adjacent the ostium.

In accordance with still another embodiment, a method is provided for delivering a stent within an ostium communicating from a main body lumen to a branch body lumen. A distal end of a delivery catheter may be advanced into the main body lumen, and one or more locator elements on the distal end may be released within the main body lumen, the one or more locator elements resiliently expanding to substantially surround the distal end. The one or more locator elements may be directed against a wall of the ostium, thereby causing one or more struts supporting the one or more locator elements to bend away from the ostium. A procedure, e.g., stent delivery, may then be performed at or within the ostium based upon the position of the one or more locator elements in the enlarged condition.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of an apparatus for delivering a stent, including a guide catheter and a delivery catheter, the delivery catheter having a distal end carrying a locator loop adjacent a balloon over which a stent is maintained.

FIG. 2 is a cross-sectional view of the catheter of FIG. 1, taken along line 2-2, with the locator loop expanded.

FIGS. 3 and 4 are cross-sectional views of alternate embodiments of delivery catheters including multiple locator loops.

FIGS. 5-10 are cross-sectional views of a patient's body, showing a method for implanting a stent within an ostium of a body lumen using the apparatus of FIG. 1.

FIGS. 11 and 12 are cross-sectional views of a patient's body, comparing a method for locating an ostium using locator loops that are disposed around a circumference of a delivery catheter with a method using one or more locator loops that are disposed on only one side of a circumference of a delivery catheter.

FIGS. 13A-13C are side, perspective, and ends views, respectively, of a locator including a single locator loop expanded transversely from a delivery catheter.

FIGS. 14A-14C are side, perspective, and ends views, respectively, of a locator including a pair of locator loops adjacent on another on one side of a delivery catheter.

FIGS. 15A-15C are perspective, and ends views, respectively, of a locator including three locator loops disposed symmetrically around a circumference of a delivery catheter.

FIGS. 16A-16C are side, perspective, and ends views, respectively, of another locator including a single locator loop having a curved tip and expanded transversely from a delivery catheter.

FIGS. 17A-17C are side, perspective, and ends views, respectively, of yet another locator including a single locator loop expanded transversely from a delivery catheter.

FIGS. 18A-18C are side, perspective, and ends views, respectively, of another locator including a “D” shaped locator loop expanded transversely from a delivery catheter.

FIGS. 19A-19C are side, perspective, and ends views, respectively, of still another locator including a “D” shaped locator loop extending transversely from a leg attached to a delivery catheter.

FIGS. 20A-20C are side, perspective, and ends views, respectively, of yet another locator including a pair of “D” shaped locator loops expanded transversely from a delivery catheter.

FIGS. 21A-21C are side, perspective, and ends views, respectively, of another locator including a narrow, curved locator loop expanded transversely from a delivery catheter.

FIGS. 22A-22C are side, perspective, and ends views, respectively, of yet another locator including a narrow, curved locator loop expanded transversely from a delivery catheter.

FIGS. 23A-23C are side, perspective, and ends views, respectively, of another locator including a pair of narrow, curved locator loop expanded transversely from a delivery catheter.

FIGS. 24A-24C are side, perspective, and ends views, respectively, of another locator including three narrow, curved locator loop expanded transversely from a delivery catheter.

FIGS. 25A-25C are side, perspective, and ends views, respectively, of a locator including a locator loop expanded transversely and asymmetrically from a delivery catheter.

FIGS. 26A-26C are side, perspective, and ends views, respectively, of another locator including a locator loop expanded transversely and asymmetrically from a delivery catheter.

FIGS. 27A-27C are side, perspective, and ends views, respectively, of still another locator including a locator loop expanded transversely and asymmetrically from a delivery catheter.

FIGS. 28A-28C are side, perspective, and ends views, respectively, of a locator including a pair of locator loops expanded transversely and asymmetrically from a delivery catheter.

FIGS. 29A-29C are side, perspective, and ends views, respectively, of a locator including three locator loops expanded transversely and asymmetrically from a delivery catheter.

FIGS. 30A-30D are perspective views of a locator loop being deployed from a guide catheter, the locator loop automatically rotating about a longitudinal axis during deployment.

FIGS. 31A-31D are perspective views of a locator loop being deployed from a guide catheter as the apparatus is advanced into an ostium.

FIG. 32 is a perspective view of another embodiment of a delivery catheter including an expandable frame adjacent a stent balloon, with the frame expanded to provide a locator loop.

FIG. 33A is a perspective view of yet another embodiment of a delivery catheter including an expandable frame adjacent a stent balloon, with the frame expanded to provide a locator loop.

FIG. 33B is a perspective detail of the frame of the delivery catheter of FIG. 33A.

FIG. 33C is an end view detail of the frame of the delivery catheter of FIG. 33A.

FIGS. 34A-34F are cross-sectional views of a patient's body, showing a method for implanting a stent using the delivery catheter of FIG. 32.

FIG. 35 is a perspective view of an expanded locator loop that may be provided on a delivery catheter.

FIGS. 36A-36C are top views of alternate patterns that may be cut from a tube to provide the locator loop of FIG. 35, the pattern being shown flat for clarity.

FIG. 37 is a top view of another pattern including a portion cut from a tube and a wire portion used to provide the locator loop of FIG. 35, the pattern being shown flat for clarity.

FIGS. 38A-38D are top views of additional patterns that may be cut from a tube to provide the locator loop of FIG. 35, the pattern being shown flat for clarity.

FIGS. 39A and 39B are side views of another embodiment of a stent delivery catheter including a plurality of locator arms adjacent the stent that are movable between collapsed and expanded configurations, respectively.

FIGS. 40A-40C are details of the stent delivery catheter of FIGS. 39A and 39B, showing different balloon and stent configurations that may be provided.

FIG. 41 is a detail of a tip that may be provided on ends of the locator arms of the delivery catheter of FIGS. 39A and 39B.

FIGS. 42A and 42B are details of alternative tips that may be provided on ends of the locator arms of the delivery catheter of FIGS. 39A and 39B.

FIGS. 43A-43D are side views of another locator arm configuration that may be provided on a delivery catheter.

FIGS. 44A and 44B are details of yet another tip that may be provided on ends of the locator arms of the delivery catheter of FIGS. 39A and 39B.

FIGS. 45A-45F are cross-sectional views of a patient's body, showing a method for implanting a stent in an ostium using a locator device including a balloon on a guide catheter.

FIGS. 46A and 46B are side views of another embodiment of a locator device including a balloon-expandable braid on a guide catheter movable between collapsed and expanded configurations, respectively.

FIG. 47 is a side view of an alternate embodiment of the locator device of FIGS. 46B.

FIGS. 48A and 48B are cross-sectional views of a patient's body, showing a method for accessing an ostium using the locator device of FIG. 47.

FIGS. 49A and 49B are side views of another embodiment of a locator device including a plurality of arms on a guide catheter that are movable between collapsed and expanded configurations, respectively, by retracting an overlying sheath.

FIGS. 50A and 50B are side views of another embodiment of a locator device including a plurality of arms on a guide catheter that are movable between collapsed and expanded configurations, respectively, by inflating an underlying balloon.

FIGS. 51A and 51B are side views of yet another embodiment of a locator device including a plurality of arms on a guide catheter that are movable between collapsed and expanded configurations, respectively, by retracting an overlying sheath.

FIG. 51C is a detail of the locator device of FIGS. 51A and 51B, showing an exemplary arm extending through a slit in the sheath.

FIGS. 52A-52C are details showing alternate tips that may be provided on the arms of the locator devices shown in FIGS. 49A-51C.

FIGS. 53A and 53B are side views of yet another embodiment of a locator device including an expandable braid on a guide catheter that is movable between collapsed and expanded configurations, respectively.

FIGS. 54A and 54B are cross-sectional details, showing alternative constructions for a tip of the locator device of FIGS. 53A and 53B.

FIGS. 55A and 55B are side views of still another embodiment of a locator device including a plurality of expandable splines on a guide catheter that are movable between collapsed and expanded configurations, respectively.

FIGS. 56A and 56B are side views of yet another embodiment of a locator device including a plurality of expandable splines or arms on a guide catheter that are movable between collapsed and expanded configurations, respectively.

FIGS. 57A-57C are side views of another embodiment of a locator device including a plurality of everting wires that are deployable from a guide catheter.

FIGS. 58A-58F are perspective views of a locator loop being deployed from a guide catheter, the locator loop automatically rotating about a longitudinal axis during deployment.

### DETAILED DESCRIPTION OF THE PREFERRED EMBODIMENTS

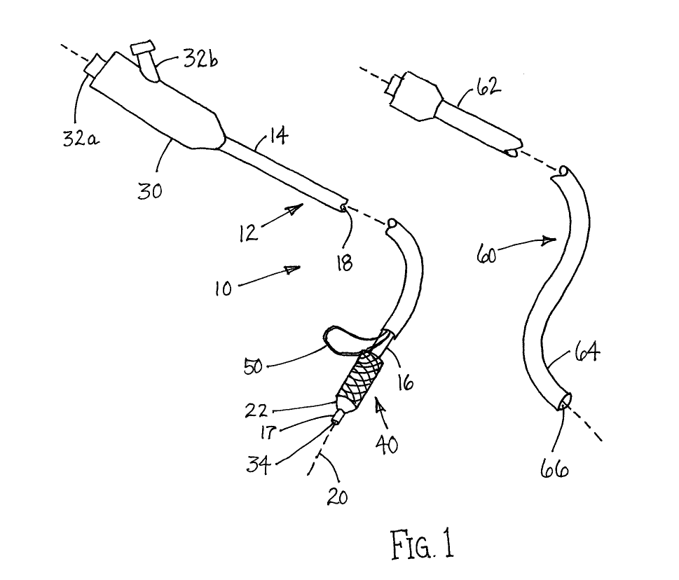

Turning to the drawings, FIG. 1 shows an exemplary embodiment of an apparatus 10 for delivering a stent or other prosthesis 40, e.g., into an ostium or other bifurcation between a main lumen and a branch lumen (not shown). Generally, the apparatus 10 includes a catheter or other elongate tubular member 12 having a proximal end 14, a distal end 16, and one or more lumens 18 extending between the proximal and distal ends 14, 16, thereby defining a longitudinal axis 20 between the proximal and distal ends 14, 16. The delivery catheter 12 includes a locator loop 50 on the distal end 16, e.g., proximal or otherwise adjacent to a stent 40 also carried on the distal end 16, which may be any of the locator loops described herein. Optionally, one or more balloons or other expandable members 22 may be provided on the distal end 16 of the delivery catheter 12 for expanding and/or deploying the stent 40, as described further below.

In addition, the apparatus 10 may include a guide catheter 60 including a proximal end 62, a distal end 64, and a lumen 66 extending therebetween. The distal end 64 may be sized and/or shaped to facilitate advancement into a patient's vasculature or other body lumen, as described further below. The lumen 66 may have sufficient size for receiving the distal end 16 of the delivery catheter 12 therethrough, e.g., with the locator loop 50 in a contracted condition, also as explained further below. Optionally, the distal end 64 of the guide catheter 60 may be biased to a predetermined shape, e.g., a “J” shape, which may facilitate positioning the guide catheter 60 within or adjacent an ostium. The guide catheter 60 may be constructed from substantially flexible and/or floppy materials, e.g., plastic having a braid or other reinforcement (not shown) that sufficiently supports the guide catheter 60 to prevent kinking or buckling, while allowing the guide catheter 60 to be directed easily through tortuous anatomy. Optionally, the apparatus 10 may include other components to provide a system or kit for delivering the stent 40, e.g., a sheath that may be advanced over and/or retracted from the distal end 16 of the delivery catheter 12, one or more syringes or other sources of inflation media and/or vacuum, tubing, and/or one or more guidewires (all not shown).

With continued reference to FIG. 1, the delivery catheter 12 may be formed from one or more tubular bodies, e.g., having variable flexibility along its length. For example, the distal end 16 may be substantially flexible to facilitate insertion through tortuous anatomy, e.g., terminating in a rounded, tapered, and/or other substantially atraumatic distal tip 17. The distal end 16 may be sized and/or shaped for introduction into a body lumen, e.g., having a diameter between about one and seven millimeters (1-7 mm), or less than 1.5 millimeters. The proximal end 14 may be substantially flexible or semi-rigid, e.g., having sufficient column strength to facilitate advancing the distal end 16 through a patient's vasculature by pushing on the proximal end 14. The delivery catheter 12 may be formed from plastic, metal, or composite materials, e.g., a plastic material having a wire, braid, or coil core, which may preventing kinking or buckling of the catheter 12 during advancement.

As shown in FIG. 1, the delivery catheter 12 may include a handle 30 on the proximal end 14, e.g., to facilitate manipulating the delivery catheter 12. The handle 30 may include one or more side ports 32 communicating with respective lumens 18 within the delivery catheter 12. The handle 30 may be molded, machined, or otherwise formed from plastic, metal, or composite material, e.g., providing an outer casing, which may be contoured or otherwise shaped to ease manipulation. The proximal end 14 of the delivery catheter 12 may be attached to the handle 30, e.g., by bonding, cooperating connectors, interference fit, and the like. Optionally, if the apparatus includes any actuatable components (not shown) on the distal end 16, the handle 30 may include one or more actuators (not shown), such as one or more slides, dials, buttons, and the like, for actuating or otherwise manipulating the components on the distal end 16 from the proximal end 14, as explained further below.

In the embodiment shown in FIG. 1, the delivery catheter 12 includes at least two lumens 18 extending between the proximal ends 14, 16. For example, the delivery catheter 12 may include a guidewire or instrument lumen that extends from a port 32a in the handle 30 to an opening 34 in the distal tip 17. The instrument lumen may have sufficient size to allow a guidewire or other rail or instrument (not shown) to be inserted therethrough, e.g., to facilitate advancing the delivery catheter 12 over the rail, as explained further below. Optionally, the handle 30 may include one or more seals (not shown) within or adjacent the port 32a, e.g., e.g., a hemostatic seal that prevents fluid, e.g., blood, from flowing proximally out of the port 32a, yet allows one or more instruments to be inserted therethrough and into the instrument lumen.

In addition, the delivery catheter 12 may include one or more inflation lumens that extend from respective side port(s) 32b in the handle 30 through the delivery catheter 12 to openings (not shown) that communicate with an interior of a respective balloon 22. The side port(s) 32b on the handle 30 may include connectors, e.g., a luer lock connector (not shown), one or more seals (also not shown), and the like. A source of inflation media and/or vacuum, e.g., a syringe filled with saline (not shown), may be connected to the side port(s) 32b, e.g., via tubing (also not shown), for expanding and/or collapsing the balloon 22.

As shown in FIG. 1, the delivery catheter 12 includes one balloon 22 on the distal end 16. Alternatively, the delivery catheter 12 may include multiple balloons (not shown) on the distal end 16 over which the stent 40 may be placed. Additional information on multiple balloon catheters and methods for using them are disclosed in co-pending application Ser. No. 11/136,266, filed May 23, 2005, and provisional application Ser. No. 60/745,177, filed Apr. 19, 2006. The entire disclosures of these references are expressly incorporated by reference herein.

The balloon (or balloons, not shown) 22 may be bonded or otherwise secured to the distal end 16 of the delivery catheter 12. For example, ends of the balloon 22 may be attached to the distal end 16 using one or more of bonding with an adhesive, sonic welding, an annular collar or sleeve, and the like. The balloon 22 may be expandable from a contracted condition (not shown, see, e.g., FIG. 6), which may facilitate advancement through a patient's vasculature, to an enlarged condition for expanding or otherwise deploying the stent 40.

The balloon 22 may be formed from substantially inelastic material, e.g., PET, nylon, or PEBAX, such that the balloon 22 expands to a predetermined size in its enlarged condition once sufficient fluid is introduced into the interior of the balloon 22. Alternatively, the balloon 22 may be formed from substantially elastic material, e.g., silicone, polyurethane, or polyethylene, such that the balloon 22 may be expanded to a variety of sizes depending upon the volume and/or pressure of fluid within the interior.

The stent 40 may be formed from a variety of materials that may be plastically deformed to allow expansion of the stent 40. For example, the stent 40 may be formed from metal, such as stainless steel, tantalum, MP35N, Niobium, Nitinol, and L605, plastic, or composite materials. In particular, the materials of the stent 40 may be plastically deformed under the pressures experienced when the balloon 22 is expanded such that all or one or more portions of the stent 40 are deformed beyond their elastic limit. Thus, when the balloon 22 is subsequently collapsed, the stent 40 may maintain its expanded configuration with minimal recoil. For example, the stent 40 material may resist collapsing back towards its reduced configuration if the tissue surrounding the body lumen attempts to constrict or otherwise return to its occluded shape.

Alternatively, at least a portion of the stent 40 may be self-expanding. For example, the stent 40 may be biased to expand at least partially outwardly yet may be constrained over the balloon 22 in a contracted condition to facilitate delivery, e.g., using a sheath, filament, and the like (not shown). In this alternative, the stent 40 may be formed from Nitinol or other shape memory or superelastic materials. Optionally, the resistance of the stent 40 to expansion may be varied along its length. This performance of the stent 40 may be based upon mechanical properties of the material, e.g., which may involve heat treating one or more portions of the stent 40 differently than other portions. In addition or alternatively, the structure of the stent 40 may be varied, e.g., by providing struts, fibers, or other components in different portions having different widths, thicknesses, geometry, and the like.

The stent 40 may be a generally tubular structure, e.g., including openings in a tubular wall that facilitate expansion of the stent 40 and/or allow tissue ingrowth. For example, the stent may be an elongate tube that has slots or other openings formed in the tube wall, e.g., by laser cutting, mechanical cutting, chemical etching, machining, and the like. Alternatively, the stent 40 may be a braided or other structure, e.g., formed from one or wires or other filaments braided or otherwise wound in a desired manner. Additional possible stent structures may include helical coil wires or sheets. If desired, one or more portions of the stent 40 may include a membrane, film, or coating (not shown), e.g., to create a nonporous, partially porous, or porous surface between cells of the stent 40 and/or to carry one or more therapeutic compounds. Additional information on stents that may be delivered using the catheter 12 may be found in co-pending application Ser. No. 60/683,920, filed May 23, 2005, 60/710,521, filed Aug. 22, 2005, 60/731,568, filed Oct. 28, 2005, 60/757,600, filed Jan. 9, 2006, 60/743,880, filed Mar. 28, 2006, and 60/745,177, filed Apr. 19, 2006. The entire disclosures of these references are expressly incorporated by reference herein.

With additional reference to FIGS. 13A-13C, in one embodiment, the locator loop 50 is an expandable member including first and second ends 52 fixed to the distal end 16 of the delivery catheter 12, first and second resilient struts 54 extending from the first and second ends 52, respectively, and a curved intermediate region 56 extending between the first and second struts 54. The locator loop 50 may formed from a single strand extending from the first end 52, through the first struts 54, the intermediate region 56, and the second strut 54 to the second end 52. Alternatively, the locator loop 50 may be formed from multiple strands that are wound about one another to form a braided or other structure. In another alternative, the locator loop 50 may include different sections of material for one or more regions of the locator loop 50 that are attached to one another, e.g., by bonding, melting, or fusing the ends, using connector bands, and the like (not shown). In yet another alternative, the locator loop 50 may be formed from a tube that has portions removed, e.g., similar to the construction of the stent 40, as described further below.

The locator loop 50 may be formed from an elastic or superelastic material, e.g., metal such as Nitinol, stainless steel, and the like, plastic, and/or composite materials (e.g., a metal wire core covered with a plastic coating). The locator loop 50 is generally resiliently compressible to a contracted condition, and biased to expand to an enlarged condition, such as that shown in FIGS. 13A-13C, when free from external forces.

For example, the locator loop 50 may be compressed against the distal end 16 of the delivery catheter 12 and constrained in the contracted condition, e.g., when the distal end 16 of the delivery catheter 12 is loaded into the lumen 66 of the guide catheter 60. In this condition, the struts 54 may extend substantially axially along the distal end 16 and the intermediate region 56 may be partially straightened, twisted, or otherwise compressed towards the surface of the distal end 16. Alternatively, a sheath (not shown) may be provided that extends over the distal end 16 of the delivery catheter 12 to constrain the locator loop 50 (and/or cover the stent 40 and balloon 22). When the distal end 16 of the delivery catheter 12 is advanced beyond the distal end 64 of the guide catheter 60 (or the overlying sheath is retracted), the locator loop 50 may resiliently expand to the enlarged condition.

The ends 52 of the locator loop 50 may be attached or otherwise secured to the distal end 16 of the delivery catheter 12. For example, an adhesive, sonic welding, fusing, and the like may be used to bond the ends 52 to the surface of the distal end 16. In addition or alternatively, a band of material, e.g., a heat shrink tube or other band of plastic, metal, wire, and the like, may be wrapped or otherwise extend around the ends 52 of the locator loop 50. In addition or alternatively, the ends 52 of the locator loop 50 may be at least partially embedded into the delivery catheter 12, e.g., into slots or holes partially or completely penetrating the wall of the delivery catheter 12. In yet another alternative, the ends 52 may be part of an annular band that may crimped or otherwise secured around the delivery catheter 12, e.g., in addition to or instead of the other attachment methods described above.

In the embodiment shown in FIGS. 13A-13C, the struts 54 are relatively short, and the intermediate region 56 is relatively long, although in some embodiments described herein, the struts 54 may be substantially longer than the intermediate region 56. The intermediate region 56 is generally curved, e.g., defining an arcuate shape approximating a portion of an ellipse or circle. Thus, the intermediate region 56 may generally define a surface, which may be substantially planar as shown in FIGS. 13A-13C or curved, as shown and described elsewhere herein. Alternatively, the intermediate region 56 may be biased to assume a more complicated curved geometry, as described further below.

The struts 54 and/or intermediate region 56 may also be shaped such that the intermediate region 56 extends transversely relative to the longitudinal axis 20 of the catheter 12. For example, the struts 54 may be curved or otherwise transition from an axial direction to a transverse direction. As shown, the ends 52 may extend substantially axially, while the intermediate region 56 extends substantially perpendicular to the longitudinal axis 20. In alternative embodiments, such as those described elsewhere herein, the intermediate region 56 and/or other portions of the locator loop 50 may extend laterally relative to the longitudinal axis 20, e.g., defining an acute or oblique angle with the longitudinal axis 20.

The locator loop 50 may have sufficient strength (e.g., column strength and/or bending resistance) to be self-supporting, yet be at partially deflectable, e.g., to provide tactile feedback to a user, as explained further below. For example, one or more portions of the locator loop 50, e.g., the struts 54 and/or intermediate region 56, may bend or flex when the locator loop 50 contacts and is pushed against a surface (e.g., a wall of a body lumen adjacent an ostium). The initial contact may provide a first tactile feedback, and thereafter resist further bending or flexing to provide a second or additional tactile feedback, as described further elsewhere herein.

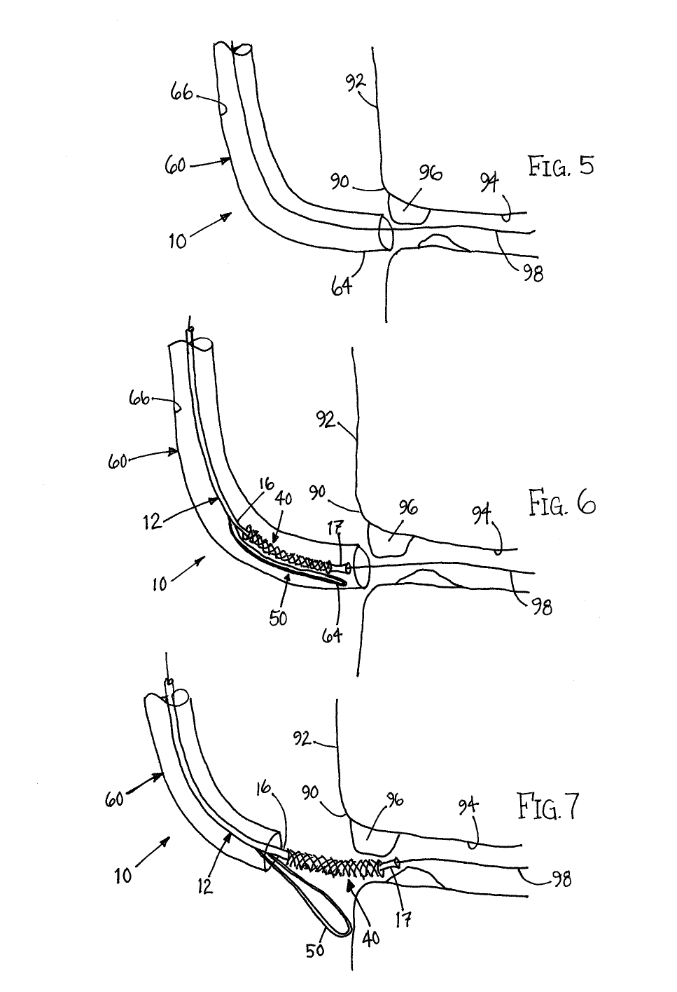

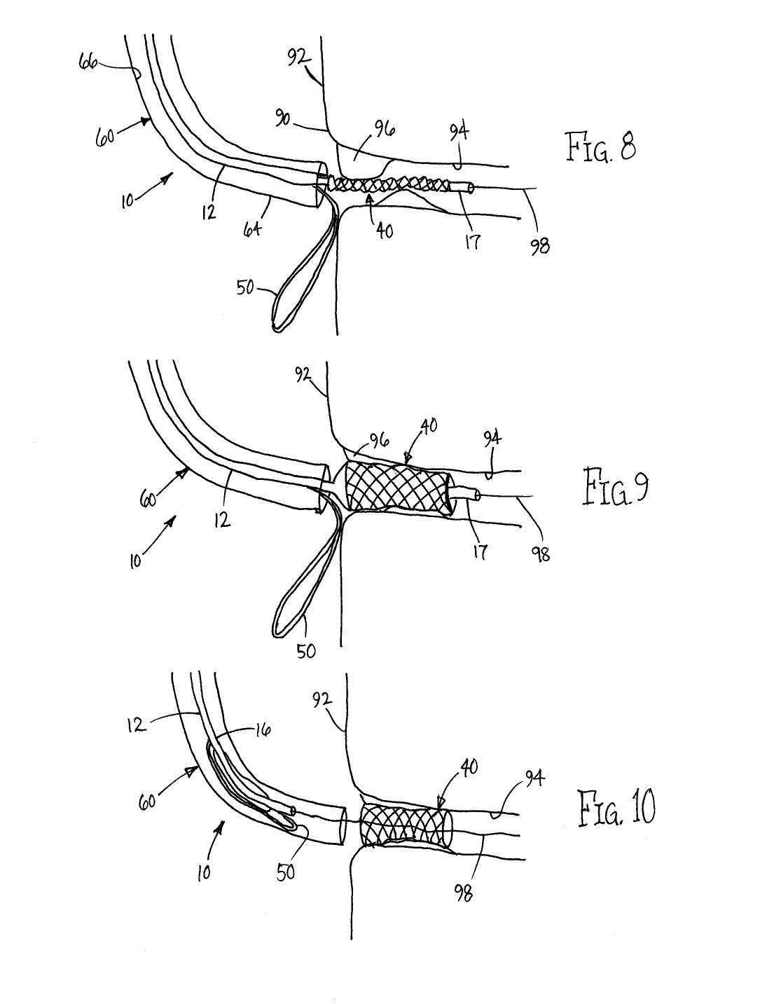

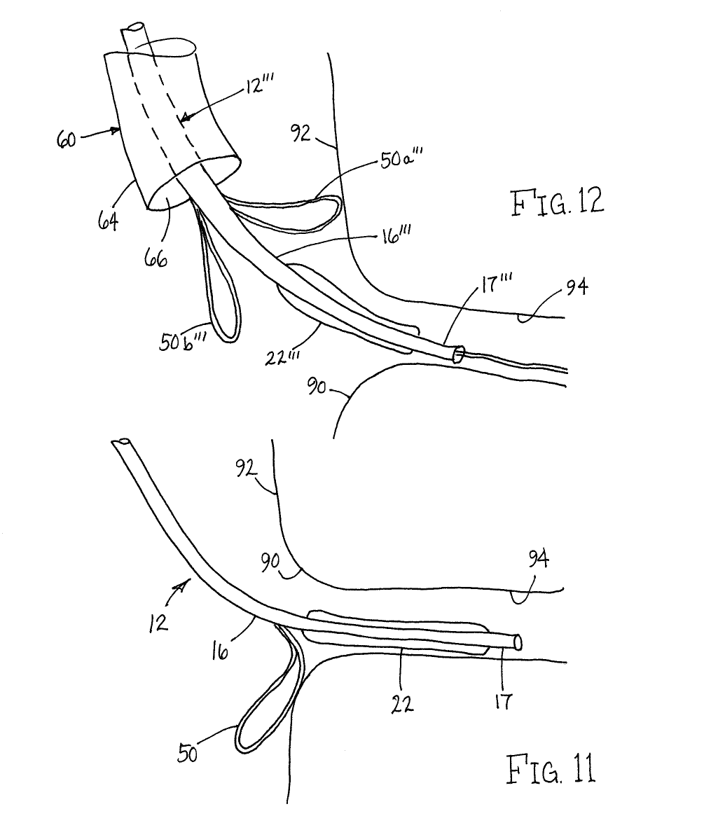

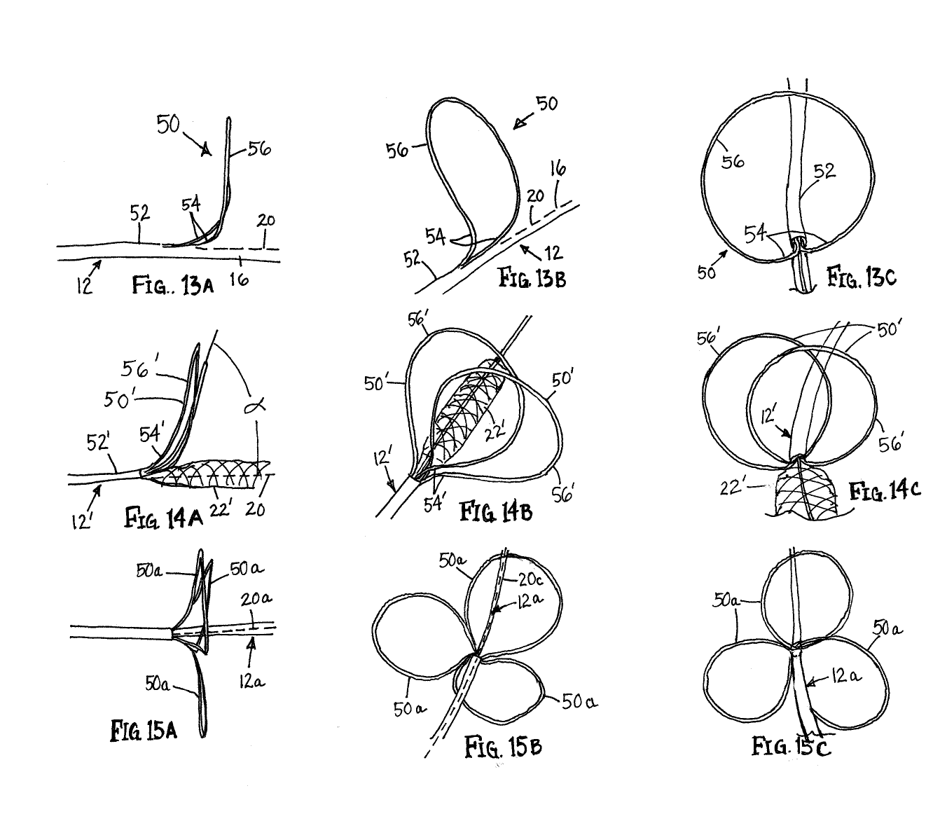

Turning to FIGS. 5-10, an exemplary method is shown for using the apparatus 10 to deliver a stent 40 into an ostium 90. The ostium 90 may be an opening in a wall of a first or main body lumen or trunk 92 that communicates with a second body lumen or branch 94. In an exemplary embodiment, the trunk 92 may be the aortic root and the branch 94 may be a coronary artery. In another embodiment, the trunk 92 may be the distal aorta, and the branch 94 may a renal artery or other abdominal branch. It will be appreciated that the apparatus and methods described herein may be applicable to a variety of bifurcations or branches that extend transversely, e.g., laterally (for example, at relatively shallow angles) or substantially perpendicularly, from another body lumen or trunk, e.g., within a patient's vasculature or other systems.

An occlusion or other lesion 96 may exist at and/or adjacent to the ostium 90, e.g., extending at least partially into the branch 94. The lesion 96 may include atherosclerotic plaque or other material that partially or completely occludes blood or other fluid flow between the trunk 92 and the branch 94.

Initially, as shown in FIG. 5, a guidewire 98 or other rail may be introduced from the trunk 92 through the ostium 90 into the branch 94. As shown, the lesion 96 at the ostium 90 partially occludes the ostium 90 and extends into the branch 94. The guidewire 98 may be placed using conventional methods. For example, a percutaneous puncture or cut-down may be created at a peripheral location (not shown), such as a femoral artery, carotid artery, or other entry site, and the guidewire 98 may be advanced through the patient's vasculature from the entry site, e.g., alone or with the aid of guide catheter 60. If the lesion 96 completely occludes the branch 94, the guidewire 98 may be directed through the occlusion or other devices (not shown) may be advanced over the guidewire 98 or otherwise in conjunction with the guidewire 98 to create a passage through the lesion 96 for the guidewire 98.

After the guidewire 98 is directed into the branch 94 beyond the lesion 96, it may be desirable to at least partially dilate the lesion 96. For example, an angioplasty catheter (not shown) may be advanced through the guide catheter 60 and/or over the guidewire 98 into and through the lesion 96, whereupon a balloon or other element on the catheter may be expanded to at least partially dilate the lesion 96. If desired, other procedures may also be performed at the lesion 96, e.g., to soften, remove, or otherwise treat plaque or other material forming the lesion 96, before the stent 40 is implanted. After completing any such procedures, instruments advanced over the guidewire 98 may be removed.

As shown in FIG. 5, the distal end 64 of the guide catheter 60 may be advanced over the guidewire 98 into the trunk 92, e.g., until the distal end 64 is disposed adjacent or proximal to the ostium 90. The guide catheter 60 may be used to advance one or more instruments (such as those just described) over the guidewire 98 and into the trunk 92 and/or branch 94.

Turning to FIG. 6, a distal end 16 of the delivery catheter 12 may be advanced over the guidewire 98 and through the lumen 66 of the guide catheter 60 from the entry site into the trunk 92. As shown, the locator loop 50, balloon 22, and stent 40 are carried in contracted conditions through the guide catheter 60. Although the locator loop 50 may be biased to extend outwardly, the guide catheter 60 may allow the locator loop 50 to slide freely within the lumen 66 while remaining in the contracted condition. Optionally, the locator loop 50 (and/or the guide catheter 60) may include a lubricious coating to reduce friction and/or otherwise facilitate advancement through the guide catheter 60.

Turning to FIG. 7, with the distal end 16 of the delivery catheter 12 within the trunk 92, the guide catheter 60 may be withdrawn from the ostium 90 to advance the distal end 16 out of the lumen 66. As the locator loop 50 is advanced out of the guide catheter 60, the locator loop 50 may resiliently expand within the trunk 92, as shown. Optionally, if a sheath overlies the locator loop 50 and/or stent 40, the sheath may be retracted before or after deploying the distal end 16 of the catheter 12 from the guide catheter 60. In this position, the distal tip 17 of the catheter 12 may extend into the ostium 90, as shown, or may be located within the trunk 92.

Turning to FIG. 8, the delivery catheter 12 may be advanced, thereby directing the distal end 16 into the ostium 90, e.g., such that the distal tip 17 extends through the lesion 96 and into the branch 94 beyond. As the distal end 16 is advanced, the locator loop 50 contacts and is pushed against the wall of the trunk 92 surrounding or adjacent the ostium 90. This initial contact may be transmitted back to the proximal end (not shown) of the delivery catheter 12 due to the increased resistance to further advancement, thereby providing tactile feedback to the user of the location of the stent 40 relative to the ostium 90. The delivery catheter 12 may be advanced further until the locator loop 50 bends, as shown, thereby preventing further distal movement. This increased resistance provides further tactile feedback that the distal end 16 of the delivery catheter 12 is positioned at an appropriate location for deploying the stent 40.

For example, the relative location of the locator loop 50 to the stent 40 on the distal end 16 of the delivery catheter 12 may be predetermined such that the position where further distal movement is impeded by the locator loop 50 corresponds to the optimum distance into the ostium 90 and/or branch 94 for deploying the stent 40. Optionally, one or more radiopaque markers (not shown) may be provided, e.g., on one or both ends of the stent 40, on the catheter 12 or balloon 22 under one or both ends of the stent 40, and/or on the locator loop 50. In one embodiment, the locator loop 50 may be made radiopaque through the incorporation of radiopaque materials in its construction, either as an integral part of the loop wire, or as a structure attached to the loop wire. Contrast may be delivered, e.g., via the delivery catheter 12 or through the guide catheter 60 (e.g., after advancing the guide catheter 60 until the distal end 64 contacts the ostium 90), to facilitate identifying the position of the stent 40 relative to the ostium 90 under fluoroscopy or other external imaging.

Turning to FIG. 9, the stent 40 may then be deployed within the ostium 90 and/or branch 94. For example, if the delivery catheter 12 includes a single balloon 22, the balloon 22 may be inflated to expand the stent 40, e.g., within the branch 94 immediately adjacent the ostium 90 to dilate and/or otherwise treat the lesion 96. The balloon 22 may expand the stent 40 to a substantially uniform cylindrical shape as shown in FIG. 9. Alternatively, the balloon 22 may expand the stent 40 to a frusto-conical or other tapered shape, similar to that shown in FIG. 40A.

In a further alternative, the delivery catheter 12 may include multiple balloons (not shown) under the stent 40 that may be used to expand portions of the stent 40 sequentially, as described in application Serial No. 11/136,266 or the other applications incorporated by reference above. For example, a proximal balloon (not shown) may be inflated to expand a proximal portion of the stent 40, e.g., into a flared configuration, adjacent the locator loop 50. The delivery catheter 12 may be advanced distally, e.g., until the flared portion conforms or otherwise contacts the wall of the trunk 92 surrounding the ostium 90. Once the flared portion is seated, another balloon may be inflated to expand a distal portion of the stent 40 within the lesion 96 and/or branch 94.

Turning to FIG. 10, once the stent 40 is expanded and/or positioned in a desired manner, the balloon(s) 22 may be collapsed, e.g., by evacuating the inflation media using a syringe or other device (not shown) at the proximal end (also not shown) of the delivery catheter 12. With the balloon 22 collapsed, the delivery catheter 12 may be withdrawn into the guide catheter 60. Optionally, the guide catheter 60 may be advanced towards or against the ostium 90 and/or against a proximal end of the stent 40 before the delivery catheter 12 is removed. This action may facilitate withdrawing the distal end 16 (e.g., the balloon 22) back through the stent 40, e.g., without substantial risk of dislodging the stent 40 from the ostium 90 and/or branch 94.

As the distal end 16 of the delivery catheter 12 is withdrawn into the guide catheter 60, the locator loop 50 may contact the distal end 64 of the guide catheter 60 and be resiliently compressed as the delivery catheter 12 is pulled into the lumen 66. For example, the locator loop 50 may be elongated, narrowed, and/or otherwise directed inwardly towards the surface of the distal end 16 of the delivery catheter 12 as the locator loop 50 is drawn into the lumen 66 of the guide catheter 60. If the struts 54 of the locator loop 50 are rounded or are inclined distally and/or transversely, they may facilitate pulling the locator loop 50 into the guide catheter 60.

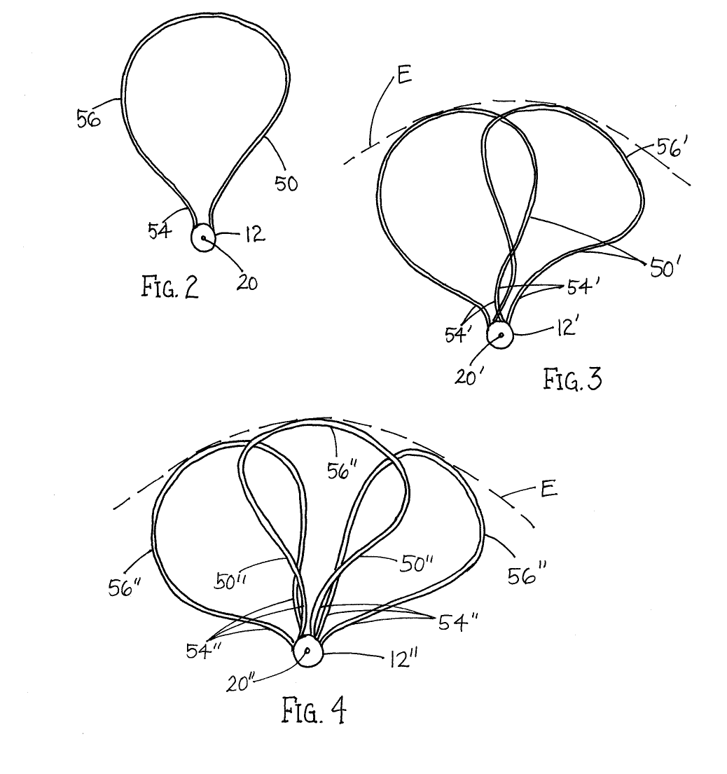

Turning to FIG. 3 (with additional reference to FIGS. 14A-14C), an alternative embodiment of a delivery catheter 12′ is shown that includes a pair of locator loops 50.′ Similar to the previous embodiment, the locator loops 50′ may be formed from a wire, e.g., one or more strands of metal, plastic, or composite material that may be deformed elastically or superelastically in a contracted condition (nor shown) and resiliently expanded to an expanded condition, as shown. The intermediate regions 56′ of the locator loops 50′ extend partially around the delivery catheter 12,′ thereby generally defining a portion of a circle or ellipse “E” about the longitudinal axis 20 of the delivery catheter 12.′ As shown in FIG. 14A, the locator loops 50′ may define an acute angle “α” between the intermediate region 56′ and the longitudinal axis 20′ towards the balloon 22.′

In a further alternative, shown in FIG. 4, a delivery catheter 12″ is shown that includes three locator loops 50″ that include intermediate regions 56″ that generally define a portion of a circle or ellipse “E” about the longitudinal axis 20.″ It will be appreciated that more than three locator loops (not shown) may be provided, if desired, that are disposed on one side of a delivery catheter. Stated differently, a plurality of locator loops may be provided asymmetrically about the longitudinal axis 20″ of the delivery catheter 12,″ e.g., such that the locator loops 50″ only define a portion of a circle or ellipse “E” about the longitudinal axis 20.″ One advantage of providing multiple locator loops on a delivery catheter is that multiple loops may distribute forces on the ostium more evenly, e.g., reducing the risk of perforation, skiving, or other damage of the wall of the ostium if the delivery catheter is pushed too forcefully.

Turning to FIGS. 11 and 12, one of the advantages that may be achieved using a delivery catheter 12 including one or more locator loops 50 disposed on one side of a delivery catheter is now described. As shown in FIG. 11, a single locator loop 50 is shown that extends transversely from one side of the delivery catheter 12 (although additional locator loops, not shown, may be disposed adjacent the single locator loop 50 shown). When the delivery catheter 12 is directed from the trunk 92 into the branch 94, there may be a substantial direction change, e.g., as much as ninety degrees (90°) or more. As the locator loop 50 contacts the ostium 90, the locator loop 50 may automatically turn the delivery catheter 12 about the longitudinal axis 20 to place the locator loop 50 on the outside bend radius of the delivery catheter 12. This may occur naturally, e.g., in order to reduce the stress on the locator loop 50. Thus, as the distal end 16 of the delivery catheter 12 is advanced into the ostium 90, the locator loop 50 may be directed against the ostium 90 on the outside bend radius. The relative location of the locator loop 50 and the stent balloon 22 may be predetermined to position the stent (not shown) within the ostium 90 and/or branch 94, as desired.

In contrast, in FIG. 12, where the delivery catheter 12′″ includes locator loops 50′″ on both sides, the locator loop 50a′″ on the inside bend radius may contact a portion of the ostium 90 before the locator loop 50b′″ on the outside bend radius does. Thus, a user may feel resistance to further distal advancement in an inconsistent manner. This may reduce the accuracy in determining the location of the ostium 90, i.e., providing the user tactile feedback before the stent 40 (not shown) on the balloon 22′″ is actually positioned desirably within the branch 94. Thus, as shown in FIG. 12, the user may feel resistance to further advancement early, and may deploy the stent too proximally due to feeling this early resistance.

Alternatively, turning to FIGS. 15A-15B, a plurality of locator loops 50a may be provided on a delivery catheter 12a that are disposed substantially symmetrically about longitudinal axis 20a. As shown, three locator loops 50a are provided, e.g., offset approximately one hundred twenty degrees (120°) from one another. Alternatively, two, four, or more locator loops (not shown) may be provided about the delivery catheter. As described further below, one advantage of a plurality of symmetrically disposed locator loops 50a is that the locator loops 50a may contact the mouth of an ostium earlier, preventing any of the locator loops from at least partially entering the ostium.

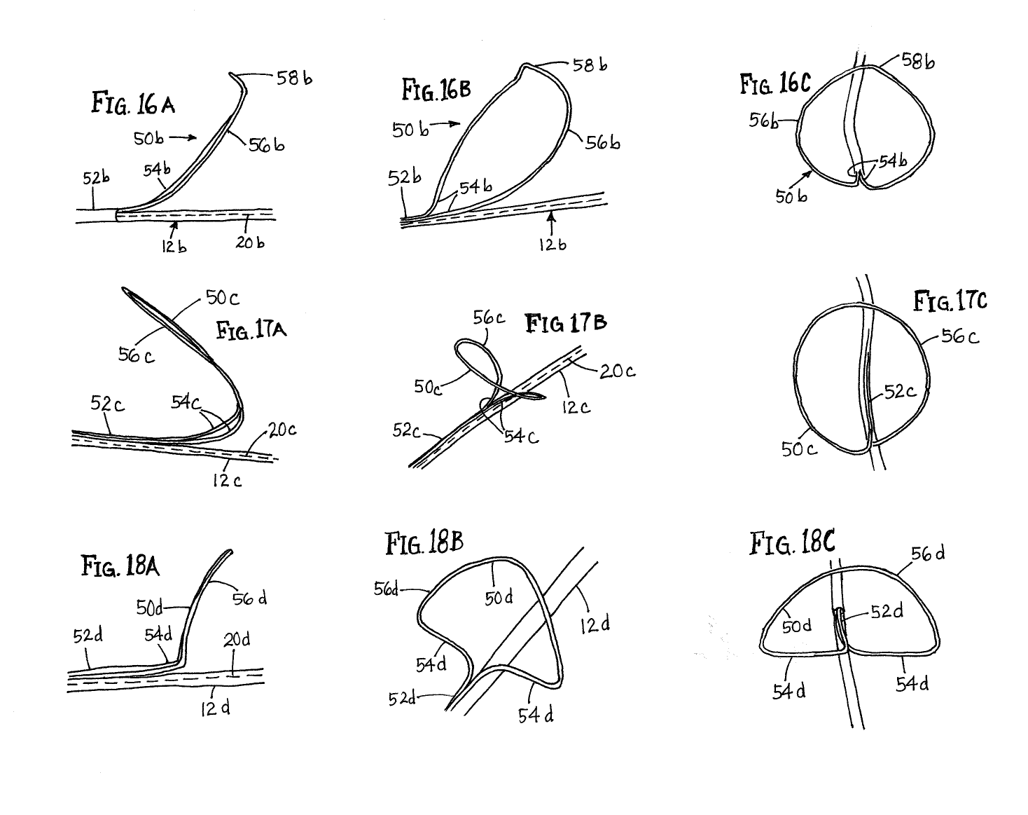

Turning to FIGS. 16A-24C, several alternative embodiments of locators are shown that may be provided on a delivery catheter, e.g., including a single locator loop (such as those shown in FIGS. 16A-19C and 21A-22C), or including a plurality of locator loops (such as those shown in FIGS. 20A-20C and 23A-24C). It will be appreciated that any of these configurations may be provided on any of the apparatus described herein individually or in sets disposed symmetrically or asymmetrically on a delivery catheter.

For example, turning to FIGS. 16A-16C, a locator loop 50b is shown that includes struts 54b that curve outwardly from ends 52b to intermediate region 56b, which defines an acute angle with the longitudinal axis 20b of the delivery catheter 12b. The intermediate region 56b includes a pointed and/or bent tip 58b, e.g., disposed substantially at a midpoint of the wire defining the locator loop 50b and/or intermediate region 56b. The intermediate region 56b generally defines a planar surface, and the bent tip 58b extends transversely from this planar surface. For example, the bent tip 58b may define an acute, substantially perpendicular, or oblique angle relative to the planar surface and/or longitudinal axis 20b. Such a bent tip 58b may reduce the risk of the locator loop 50b entering an ostium during deployment, e.g., by directing an axial force from distal advancement of the delivery catheter 12b radially outwardly away from the ostium.

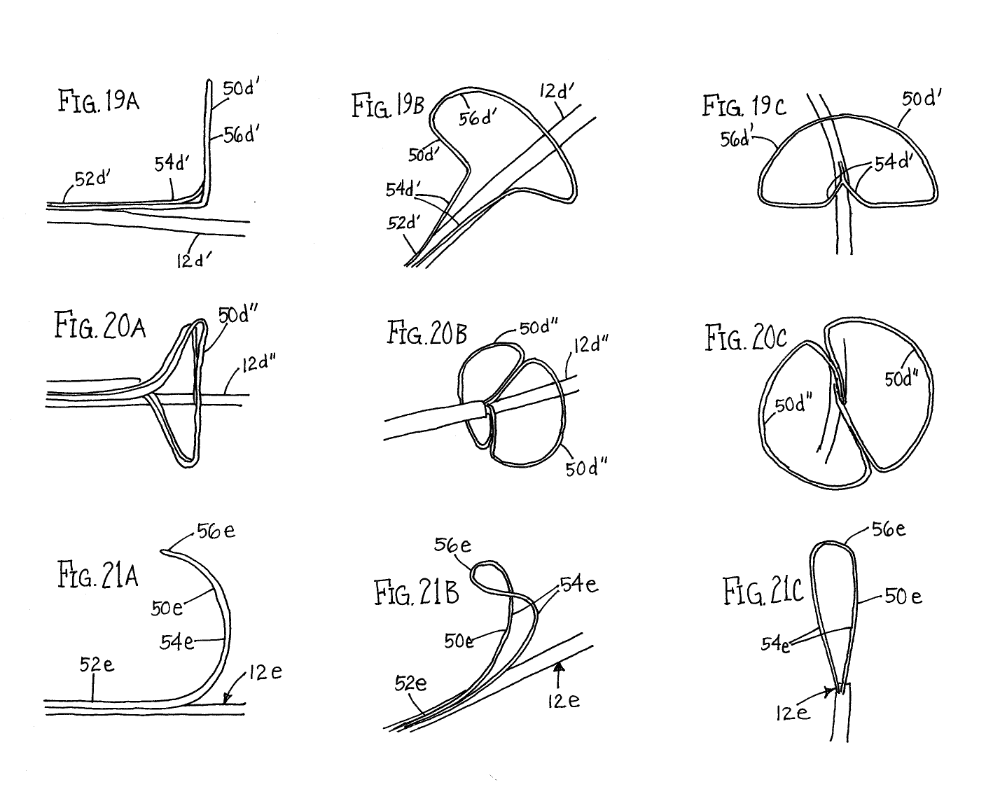

Turning to FIGS. 17A-17C, a locator loop 50c is shown that includes an intermediate region 56c that defines an oblique angle with the longitudinal axis 20c of the delivery catheter 12c. The struts 54c may curve more dramatically, i.e., extending distally and transversely relative to the longitudinal axis 20c to more quickly deploy upon being exposed within a body lumen. This may reduce the risk of the locator loop 50c being advanced into an ostium before the locator loop 50c has opened completely.

Turning to FIGS. 18A-18C, a locator loop 50d is shown that includes ends 52d fixed to delivery catheter 12d, struts 54d that extend axially initially and then bend away from one another, and a curved intermediate region 56d extending between the struts 54d. Thus, upon deployment, the locator loop 50d may define a substantially “D” shape, e.g., defining a generally planar surface between the intermediate region 56d and portions of the struts 54d. This shape may allow the locator loop 50d to distance itself more quickly from the delivery catheter 12d upon deployment. The distal and transverse angle of the struts 54d (defining an acute angle with the longitudinal axis 20d) may enhance tactile feedback and/or facilitate withdrawal of the locator loop 50d back into a guide catheter or other sheath (not shown).

Turning to FIGS. 19A-19C, a variation of the locator loop 50d′ of FIGS. 18A-18C is shown in which the struts 54d′ include a longer axial portion. This configuration may allow the locator loop 50d′ to extend at least partially over the stent and/or stent balloon (not shown for simplicity). The longer struts 54d′ may also cause the locator loop 50d′ to position the stent more proximally within an ostium, i.e., closer to the trunk than the branch. Turning to FIGS. 20A-20C, another variation is shown including a pair of locator loops 50d,″ similar to the locator loop 50d shown in FIGS. 18A-18C, disposed on opposite sides of a delivery catheter 12d.″

Turning to FIGS. 21A-21C, a locator loop 50e is shown that includes relatively long struts 54e that extend from fixed ends 52e to a relatively short radius intermediate region 56e. Thus, the locator loop 50e may have a flower petal or “banana peel” shape, which may define a curved surface, as shown, or a substantially planar surface (not shown). As shown, the struts 54e define a radius of curvature between about ninety and one hundred eighty degrees (90-180°), e.g., close to one hundred eighty degrees (180°), which may reduce the risk of the locator loop 50e being directed into an ostium as the locator loop 50e is deployed.

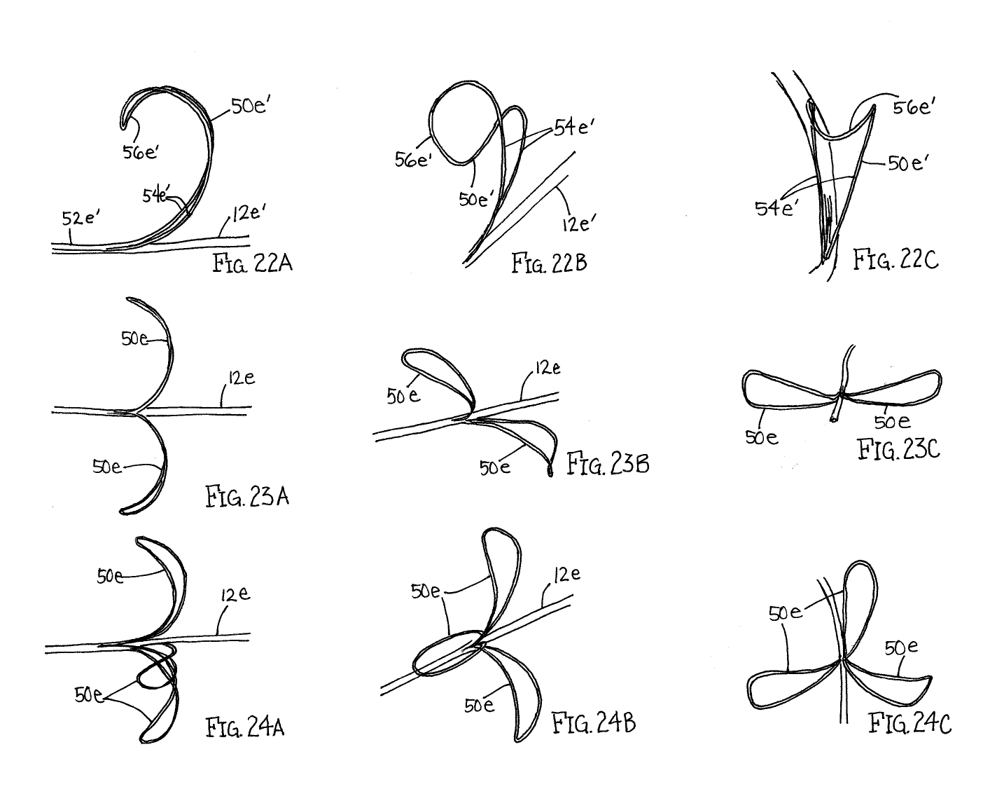

Optionally, as shown in FIGS. 22A-22C, the struts 54e′ may define a radius of curvature greater than one hundred eighty degrees (180°), e.g., approaching two hundred seventy degrees (270°). This configuration may further reduce the risk of the locator loop 50e′ being accidentally directed into an ostium during deployment. In addition or alternatively, two, three, or more such locator loops 50e may be provided on a delivery catheter 12e, as shown in FIGS. 23A-24C.

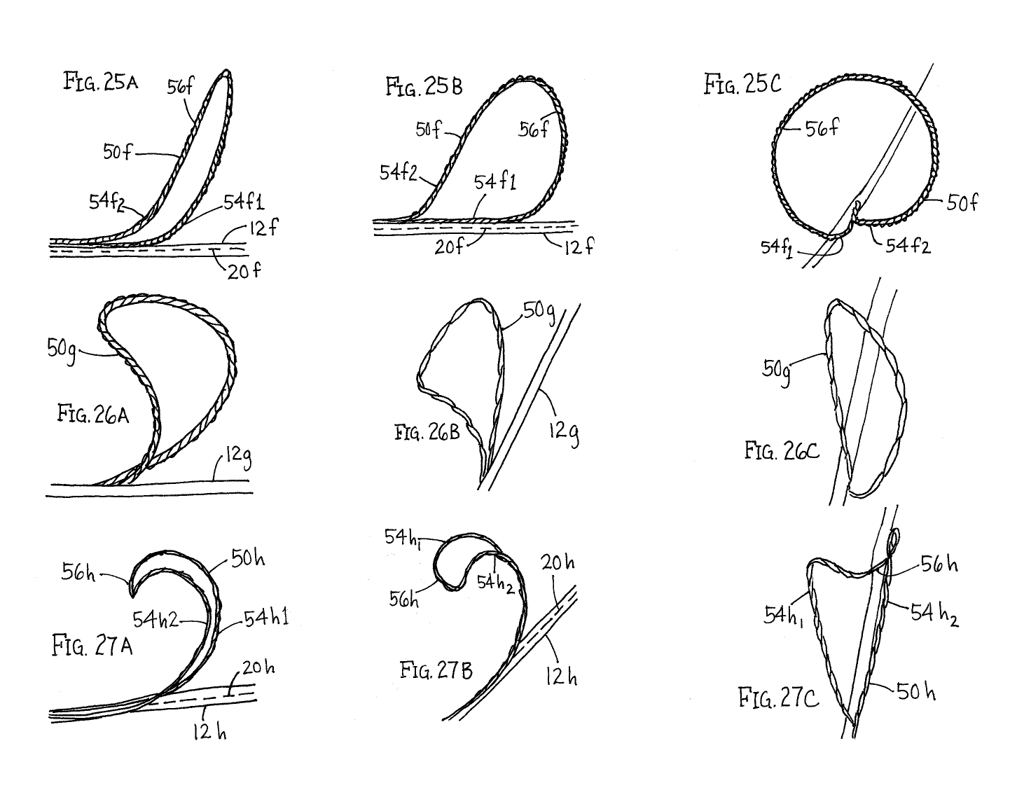

Turning to FIGS. 25A-25C, in another embodiment, a locator loop 50f may be provided on a delivery catheter 12f that is twisted asymmetrically relative to the longitudinal axis 20f of the delivery catheter 12f. Stated differently, unlike the previous embodiments, the surface defined by the locator loop 50f defines a normal axis that does not extend substantially parallel to the longitudinal axis 20f. Instead, as shown, one strut 54f1 may initially extend more axially than the other strut 54f2 such that the intermediate region 56f defines an angle that intersects the longitudinal axis 20f at a non-orthogonal angle (i.e., other than ninety degrees (90°)).

In an alternative embodiment, shown in FIGS. 26A-26C, the locator loop 50g may include multiple wires wound around each other that are arranged asymmetrically. Similarly, a multiple wire locator may be provided for any of the embodiments described herein.

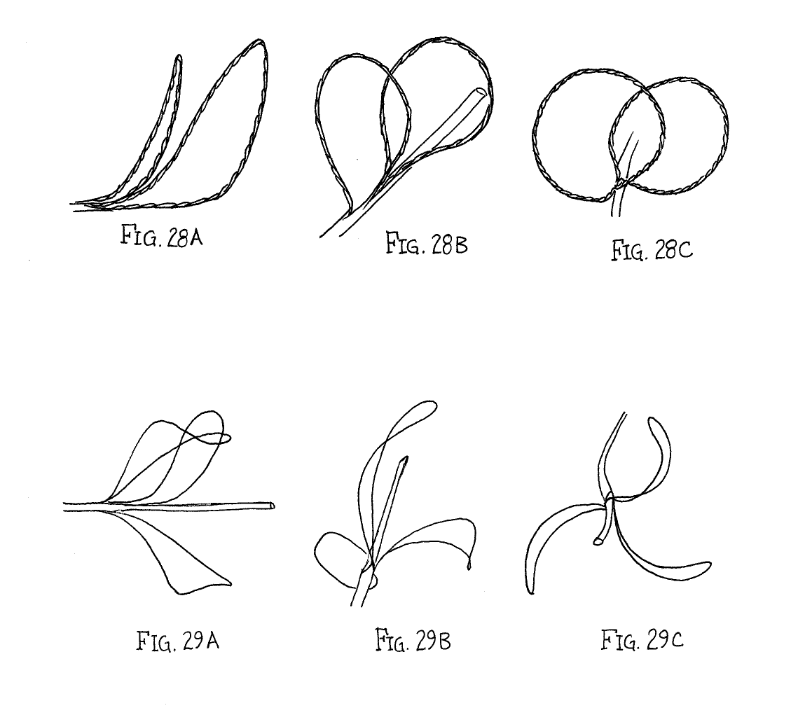

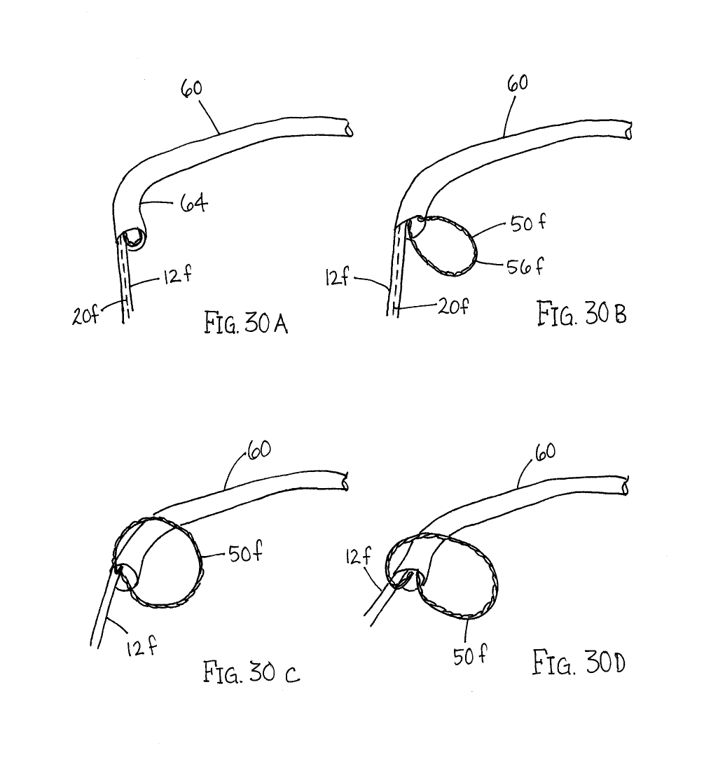

In yet another alternative embodiment, shown in FIGS. 27A-27C, the locator loop 50h may include one strut 54h1 that is longer than the other strut 54h2, thereby causing the intermediate region 56h to be disposed non-orthogonally with respect to the longitudinal axis 20h of the delivery catheter 12h. In other variations, the delivery catheter may include two (FIGS. 28A-28C), three (FIGS. 29A-29C), or optionally more (not shown) such locator loops. Thus, in these variations, the locator loop(s) may have a tendency to “twist” relative to the longitudinal axis of the delivery catheter.

Turning to FIGS. 30A-30D, with additional reference to FIGS. 25A-25C, a method is shown for deploying a delivery catheter 12f including one or more locator loops having an axial twist (one locator loop 50f shown). Initially, with reference to FIG. 30A, the locator loop 50f may be disposed within a guide catheter 60 (or other sheath, not shown). Because of the bias of the locator loop 50f to expand radially outwardly (and/or because the distal end 64 of the guide catheter 60 is generally biased into a curved shape), the locator loop 50f may be oriented within the distal end 64 of the guide catheter 60 such that the apex or intermediate region 56f of the locator loop 50f is disposed along the inside radius of the distal end 64. This location imposes the lowest stress on the locator loop 50f, e.g., being closer to its deployed, enlarged configuration. Consequently, as shown in FIG. 30B, as the intermediate region 56f of the locator loop 50f first emerges from the guide catheter 60, the locator loop 50f is generally located along the inside radius of the guide catheter 60.

Turning to FIG. 30C, as the locator loop 50f is deployed further, the less axial, more curved strut of the locator loop 50f may bear against the guide catheter 60, causing the locator loop 50f (and consequently, the distal end of the delivery catheter 12f) to twist or rotate about the longitudinal axis 20f. As shown in FIG. 30D, once the locator loop 50f is fully deployed, the locator loop 50f may have rotated substantially, e.g., by at least about sixty degrees (60°) about the longitudinal axis 20f, relative to its initial position shown in FIG. 30B.

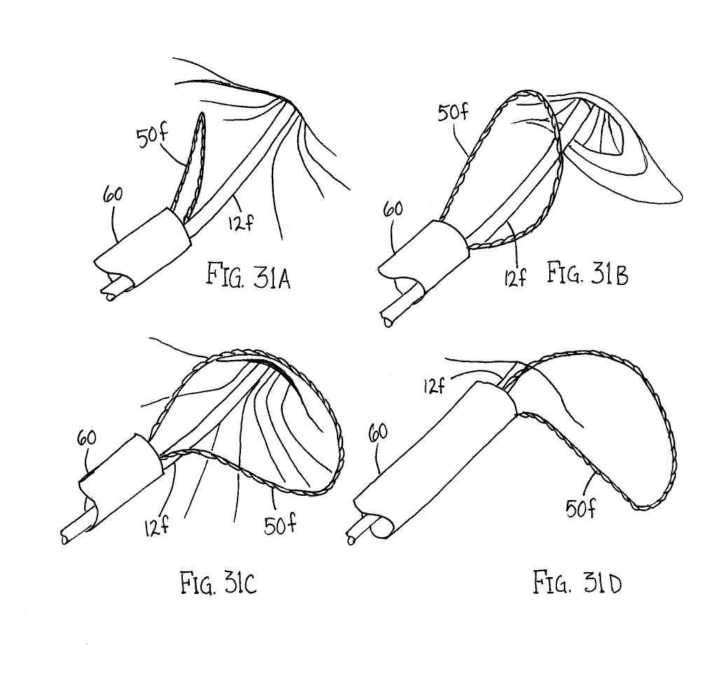

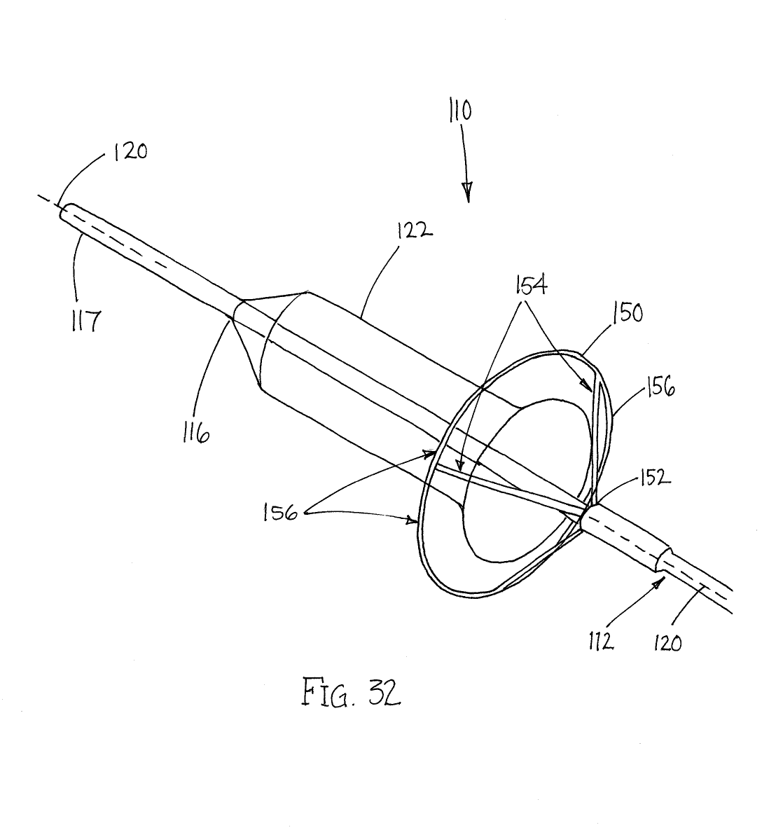

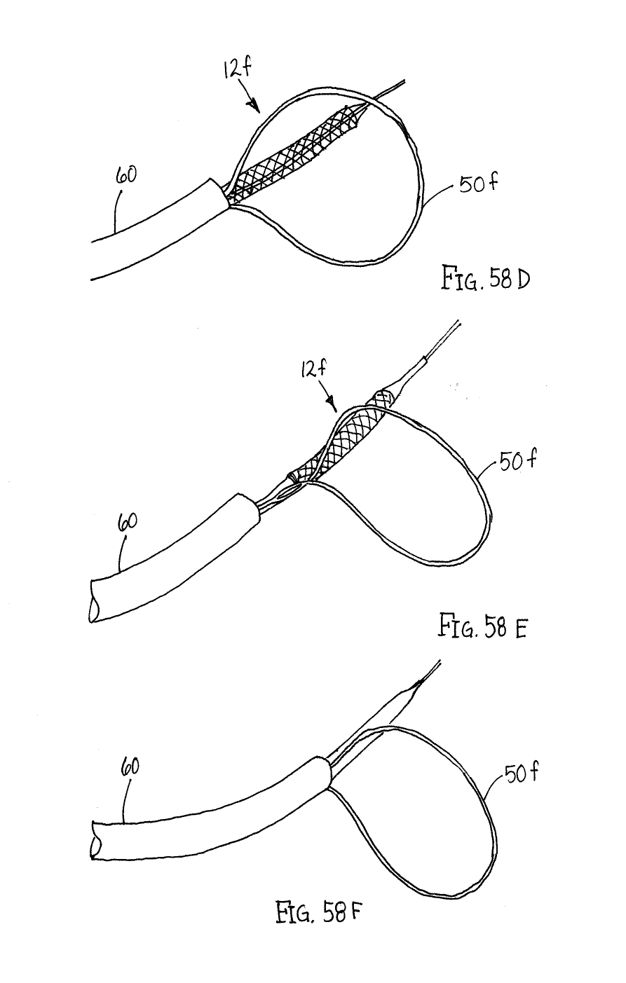

Turning to FIGS. 31A-31D, a method for using the locator loop 50f to locate and position the delivery catheter 12f relative to an ostium 90 is now described. In FIG. 31 A, the locator loop 50f initially deploys along the inside radius of the guide catheter 60, as described above. Because of the relative small initial size of the locator loop 50f as it first emerges, there is a risk that the locator loop 50f may enter the ostium along with the stent and/or stent balloon (not shown for simplicity). Turning to FIG. 31B, however, because of the bias of the locator loop 50f to twist axially, as the locator loop 50f is deployed further, the locator loop 50f (and consequentially the distal end of the delivery catheter 12f) may rotate about the longitudinal axis 20f of the delivery catheter 12f, thereby avoiding the ostium 90. FIGS. 58A-58F are additional perspective views of the delivery catheter 12f being advanced from the guide catheter 60. As the distal end is being advanced, a locator loop 50f is shown emerging from an interior of the curve defined by the guide catheter 60 (FIG. 58B). As the locator loop 50f becomes fully exposed, the locator loop 50f′ automatically rotates around the longitudinal axis, e.g., up to one hundred eighty degrees (180°) (FIGS. 58C-58F), due to the stress stored in the locator loop 50f when it is constrained within the guide catheter 60.

As shown in FIG. 31C, once the locator loop 50f rotates around and past the ostium 90, the locator loop 50f may be fully deployed in contact with the wall adjacent the ostium 90. Turning to FIG. 31D, the delivery catheter 12f may then be advanced into the ostium 90 until the locator loop 50f resists further advancement, thereby providing tactile feedback to the user that the stent may be positioned at the desired implantation site within the ostium 90 and/or branch, as described above.

Turning to FIG. 32, another embodiment of a delivery catheter 112 is shown that includes a distal end 116 carrying a locator loop 150 and a balloon 122 for delivering a stent (not shown). The delivery catheter 112 and/or balloon 122 may be constructed and used similar to other embodiments described herein. Similar to the previous embodiments, the locator loop 150 includes ends 152 attached to the distal end 116 of the delivery catheter 112, and a plurality of struts or spokes 154 extending from the ends 152 to curved outer loop regions 156. As shown, the struts 154 are offset from one another approximately one hundred twenty degrees (120°) about a longitudinal axis 120 of the delivery catheter 112, and are biased to extend distally and transversely, e.g., to define an acute angle with the longitudinal axis 120. The outer loop regions 156 extend between adjacent struts 154, thereby generally defining a circle or ellipse around the longitudinal axis 120.

In one embodiment, the locator loop 150 may be formed from multiple segments of wire, with each segment defining a first end, a first strut, a curved region to a second strut, and a second end. Thus, in the embodiment shown in FIG. 32, the locator loop 150 may include three wire segments. Optionally, the adjacent struts 154 may be at least partially attached to one another, e.g., by bonding, sonic welding, fusing the struts 154, and/or winding the struts 154 around one another. In addition, adjacent struts 154 may be coupled at least partially to one another, for example, by disposing the adjacent struts 154 within a common tubular structure. The tubular structure may extend the full length of the struts 154 or may extend only partially, e.g., adjacent a base of the struts 154. Attaching adjacent struts 154 to one another may increase a rigidity of the struts 154 compared to the loop regions 156. Optionally, only a portion of the adjacent struts 154 may be attached to one another, e.g., immediately adjacent the ends 152. Alternatively, the locator loop may include more than three wire segments (not shown), e.g., four, five, six, or more segments, although increasing the number of segments may increase the relative rigidity of the locator loop.

In another embodiment, the locator loop 150 may be cut or otherwise formed from a single section of tubing. In this embodiment, the struts 154 may include single spokes (rather than adjacent struts) offset about the longitudinal axis 120, and curved regions 156 extending between the spokes 154. Thus, the locator loop 150 may be a unitary frame including a circular or elliptical portion defined by the curved regions 156, and a plurality of spokes that couple the locator loop 150 to the delivery catheter 112.

The locator loop 150 may be formed by laser cutting, mechanically cutting, etching, or otherwise removing material from a tube to create the frame. Exemplary materials for the locator loop 150 include elastic or superelastic materials, such as Nitinol (NiTi), stainless steel, a polymer or other plastic, or other materials described elsewhere herein. Optionally, the material of the locator loop 150 may be heat treated, e.g., to bias the frame to adopt the enlarged condition shown in FIG. 32, yet allow the locator loop 150 to be radially compressed to a contracted condition (not shown) for delivery.

During use, the distal end 116 of the delivery catheter 112 may be loaded into a guide catheter (not shown) with the locator loop 150 constrained in a contracted condition using an introducer device (not shown). For example, after manufacturing or any time before use, the locator loop 150 may be compressed and placed within a tubular member or other introducer device that is small enough to be received in the proximal end of the guide catheter. Once the distal end 116 of the delivery catheter 112 and the locator loop 150 are positioned in the guide catheter, the introducer device may be removed, and the delivery catheter 112 advanced through the guide catheter, similar to the methods described above.

When the distal end 116 of the delivery catheter 112 is deployed from the guide catheter, the locator loop 150 may resiliently spring open and assume the enlarged condition shown in FIG. 32. As the distal end 116 of the delivery catheter 112 is advanced into an ostium (not shown) of a vessel to be treated, the locator loop 150 may contact the wall of the main body lumen or trunk surrounding the ostium, and prevent further movement, similar to the methods described elsewhere herein. Once the stent (not shown) is properly located using the locator loop 150, the stent may be expanded or otherwise delivered, as described elsewhere herein. The delivery catheter 112 may then be withdrawn into the guide catheter or sheath, causing the locator loop 150 to collapse as it enters the guide catheter. The entire apparatus may then be removed from the patient.

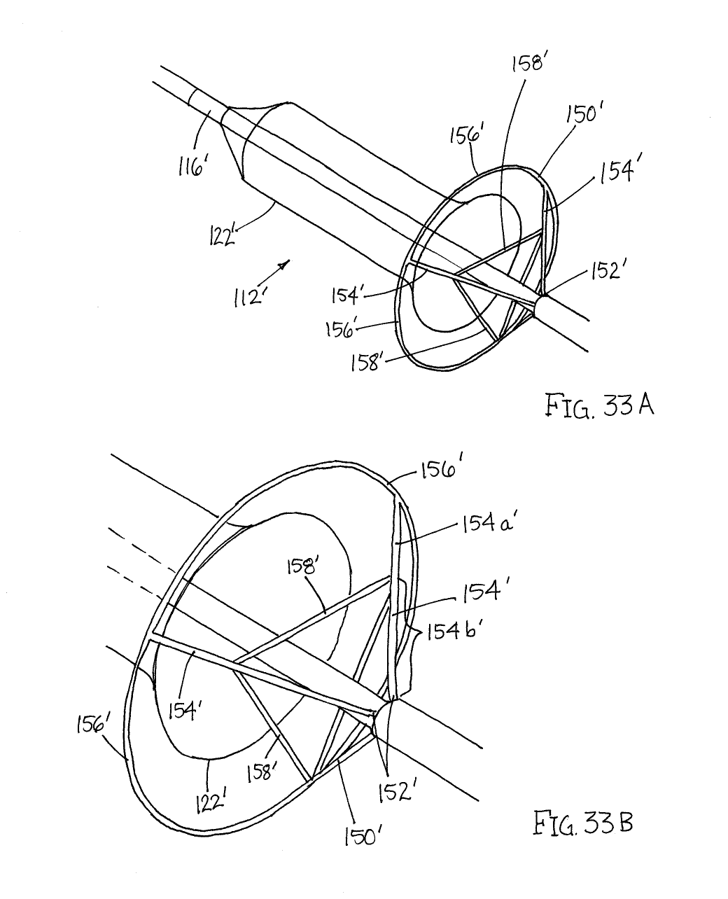

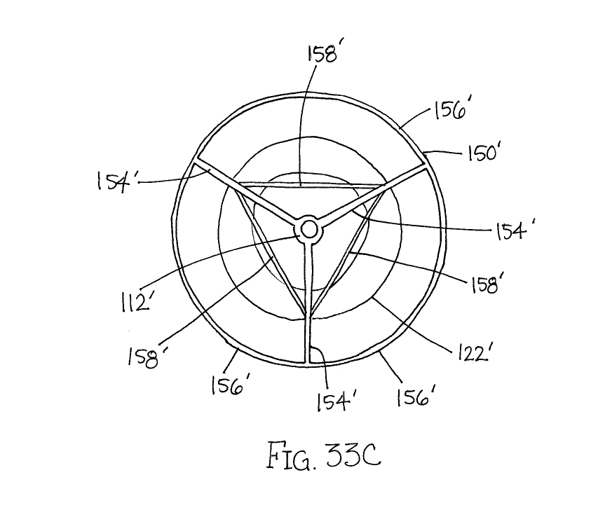

Turning to FIGS. 33A-33C, an alternative embodiment of a delivery catheter 112′ is shown that includes a balloon 122′ (or multiple balloons, not shown) and a locator loop 150′ on a distal end 116′ of the delivery catheter 112.′ The locator loop 150′ includes a plurality of struts or spokes 154′ extending between ends 152′ and curved regions 156,′ similar to the previous embodiment. Unlike the previous embodiments, the locator loop 150′ includes a plurality of supports 158′ that extend between the struts 154.′ The supports 158′ enhance a rigidity of the struts 154′ between the connection point of the supports 158′ and the ends 152′ attached to the delivery catheter 112.′ Thus, the struts 154′ may be divided into a deflectable outer portion 154a′ and a relatively rigid inner portion 154b .′

As best seen in FIG. 33C, the location where the supports 158′ connect to the struts 154′ may correspond to a maximum inflation diameter of the balloon 122.′ In addition or alternatively, the supports 158′ may generally define a diameter that is larger than a branch body vessel or ostium into which the delivery catheter 112′ may be directed, as explained further below.

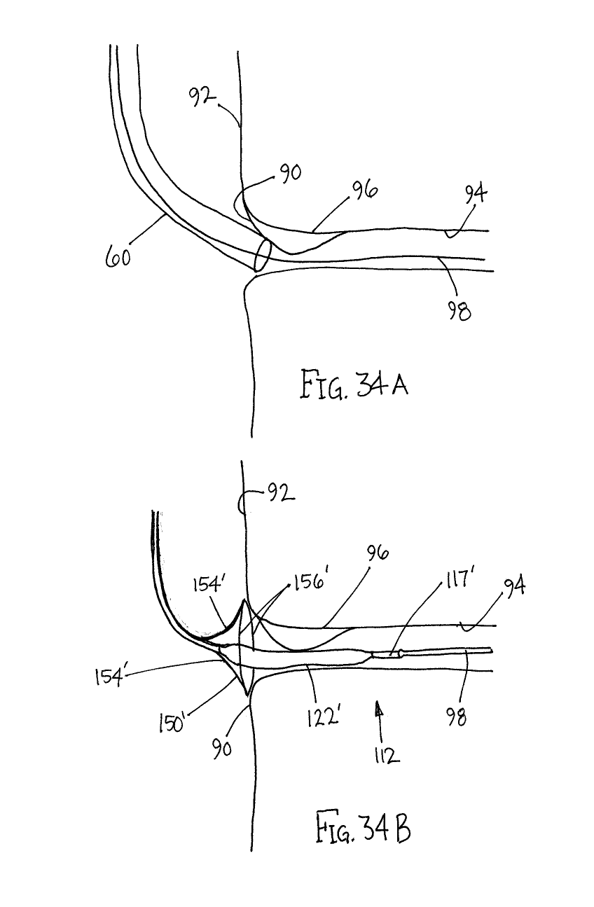

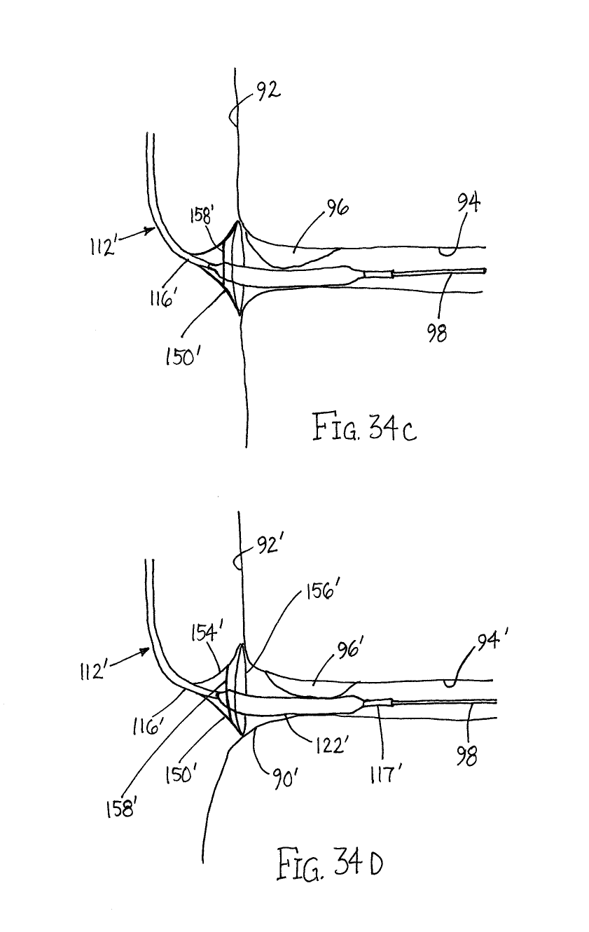

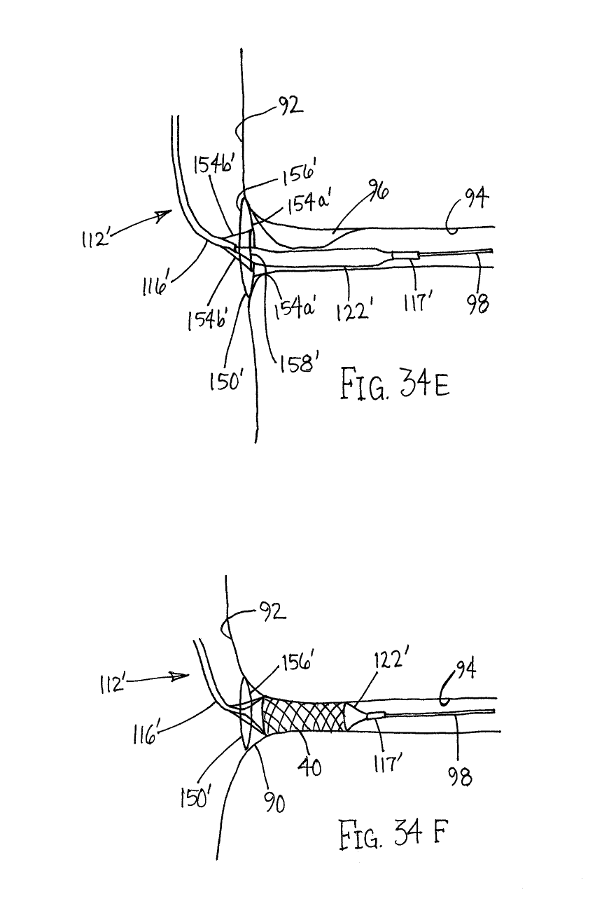

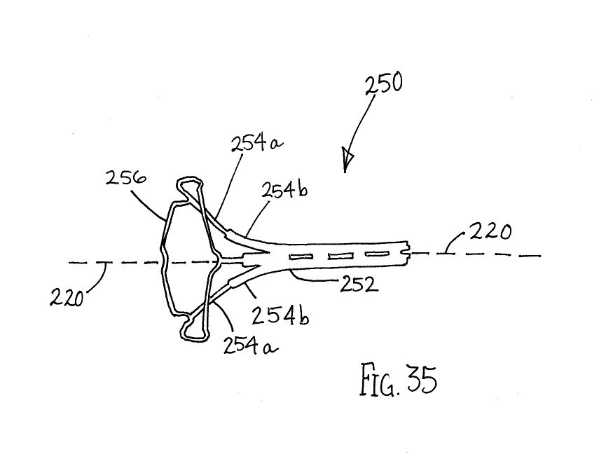

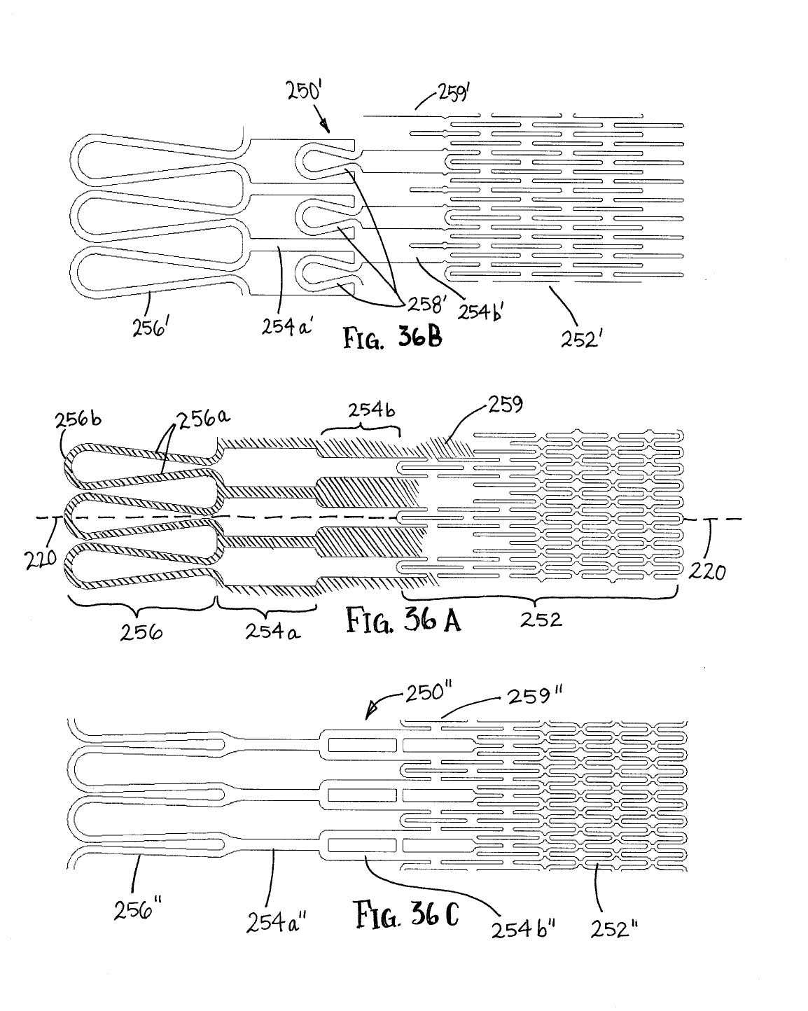

Turning to FIGS. 34A-34F, a method is shown for implanting a stent 40 using the delivery catheter 112′ of FIGS. 33A-33C. Initially, as shown in FIG. 34A, a guidewire 98 and guide catheter 60 may be placed in a main body lumen 92 and/or extending through an ostium 90 into a branch body lumen 94, which may include a lesion 96, similar to the other embodiments described elsewhere herein. Turning to FIG. 34B (where the guide catheter 60 has been omitted for clarity and/or withdrawn at least partially from the ostium 90), the distal end 116′ of the delivery catheter 112′ may be advanced into the main body lumen 92, e.g., through the guide catheter 60 (or other sheath, not shown). As the distal end 116′ is deployed from the guide catheter 60 (or other sheath), the locator loop 150′ may expand to its enlarged condition as shown (and similar to that shown in FIGS. 33A-33C).

The distal end 116′ of the delivery catheter 116′ may be advanced over the guidewire 98 into the ostium 90, e.g., until the balloon 122′ (and stent 40 carried thereon, not shown for clarity) is disposed adjacent the lesion 96 and/or within the branch 94. As shown in FIG. 34B, the curved region 156′ of the locator loop 150′ may contact the wall of the main body lumen 92 surrounding the ostium 90, thereby providing tactile feedback to the user.

One advantage of the locator loop 150′ is shown in FIGS. 34C and 34D. For example, in FIG. 34C, the ostium has a relatively shallow length, i.e., transitions more quickly from the main body lumen 92 to the branch 94. In this embodiment, the balloon 122′ and stent may be located closer to the main body lumen 92 within the ostium 90. Because of the predetermined relationship of the locator loop 150′ to the balloon 122,′ the stent may be positioned far enough into the ostium 90 without extending into the main body lumen 92. In contrast, in FIG. 34D, the ostium 90′ has a longer, more gradual transition between the main body lumen 92′ and the branch 94.′ Because of this larger transition, the locator loop 150′ may be received deeper in the ostium 90, positioning the balloon 122′ (and stent) deeper in the ostium 90.′ Thus, the size and/or shape of the locator loop 150′ may automatically position the stent at a desired depth into an ostium even if the size and/or shape of the particular ostium encountered varies.

Returning to FIG. 34B, after the locator loop 150′ contacts the wall around the ostium 90, the distal end 116′ of the delivery catheter 112′ may be advanced further into the ostium 90 and/or branch 94. This distal force causes the locator loop 150′ to flex or bend, as shown in FIG. 34E. With additional reference to FIGS. 33A-33C, because of the supports 158′ on the locator loop 150,′ the struts 154′ may be bend in the outer portion 154a′ away from the ostium 90, causing the curved regions 156′ to remain stationary, while the distal end 116′ of the delivery catheter 112′ enters further into the ostium 90. When the supports 158′ and/or the ends of the inner portions 154b′ of the struts 154′ contact the ostium 90, additional tactile feedback will be provided to the user, indicating that the delivery catheter 112′ should not (and cannot) be advanced further. This feedback informs the user (and may be confirmed using fluoroscopy or other external imaging, as described elsewhere herein) that the stent 40 is now in an appropriate position for deployment.

Turning to FIG. 34F, the balloon 122′ may then be inflated to expand the stent 40 within the ostium 90 and/or branch 94, e.g., to dilate or otherwise treat the lesion 96. Once the stent 40 is deployed, the balloon 122′ may be deflated, and the distal end 116′ retracted into the guide catheter 60 (not shown) or otherwise removed from the patient, similar to methods described elsewhere herein.

Turning to FIG. 35, another embodiment of a locator loop 250 is shown that may be formed from a section of tubing, e.g., from Nitinol or other material. As shown, the locator loop 250 includes a collar 252 from which struts 254 extend to outer loop portions 256, generally similar to previous embodiments. The outer loop portions 256 may be biased to expand to the enlarged condition shown in FIG. 35, but may be compressed or otherwise provided initially in a contracted condition.

With additional reference to FIG. 36, which shows a tube 259 unrolled about its circumference, the locator loop 250 may include distinct sections cut or otherwise formed along a length of the tube 259 to provide the different regions of the locator loop 250. For example, one end of the tube 259 may be formed into the collar 252, which may include a plurality of cells or other structure allowing the collar 252 to be crimped or otherwise secured onto a delivery catheter (not shown). Alternatively, the collar 252 may be a solid walled band that may be fitted around or otherwise to the delivery catheter. In a further alternative, the collar 252 may be eliminated and ends of the struts 254 may be attached directly to the delivery catheter, similar to embodiments described elsewhere herein.

An intermediate portion of the tube 259 may be formed into the struts 254, which extend generally axially when cut from the tube 259. Each strut 254 may include an inner portion 254b coupled to the collar 252 and an outer portion 254a coupled to the outer loop portions 256. The inner portion 254b may have a wider width than the outer portion 254a and/or may have a greater thickness (not shown) such that the inner portion 254b has a higher resistance to bending than the outer portion 254a. Stated differently, the inner portion 254b may provide a relatively stiff spoke portion, while the outer portion 254a provides a relatively flexible spoke portion. When a bending moment is applied to the struts 254, e.g., when the locator loop 250 is directed against an ostium, as described above, the struts 254 may preferentially bend at the transition between the inner and outer portions 254b, 254a. Thus, the locator loop 250 may respond and provide tactile feedback similar to the embodiments described above.

The outer loop portions 256 may be formed from the end of the tube 259 opposite the collar 252. The outer loop portions 256 may be formed as a plurality of serpentine elements that extend around a circumference of the tube between adjacent struts 254. As shown, the outer loop portions 256 include a pair of straight sections 256a extending from adjacent struts 254 and a loop 256b extending between the straight sections 256a. Alternatively, if desired, multiple loops (not shown) may be provided between adjacent struts to provide outer loop portions 256 that expand in a desired manner.

Once the tube 259 is cut, e.g., into the pattern shown in FIG. 36A, the tube may be expanded and treated, e.g., heat set, to program the flared, enlarged condition of the locator loop 250, as shown in FIG. 35. Optionally, the tube may be formed from a shape memory material, e.g., Nitinol, such that the tube may be heat set to the enlarged condition in an austenitic state, and cooled to a martensitic state where the locator loop 250 may be plastically deformed back into the contracted condition. Subsequently, when the locator loop 250 is heated, e.g., to body temperature within a patient, the locator loop 250 may transition back to its austenitic state, whereupon the locator loop 250 may be biased to return to the enlarged condition when deployed, as described above. Before or after heat treatment, the locator loop 250′ may be cleaned or otherwise treated, e.g., using electro-polishing, abrasive blasting, and/or pickling.

Turning to FIG. 36B, an alternative embodiment of a locator loop 250′ is shown that includes a collar 252′, a plurality of struts 254,′ and a plurality of outer loop portions 256′ similar to the previous embodiment. Unlike the previous embodiment, the locator loop 250′ may include a plurality of loops 258′ that extend between adjacent struts 256,′ e.g., at the transitions between the inner and outer portions 254b, 254a of the struts 254.′ Upon deployment, the loops 258′ may at least partially straighten to provide supports between the struts 254,′ thereby reinforcing the inner portions 254b from bending when the locator loop 250′ is directed against an ostium. Thus, the loops 258′ may become transverse supports, similar to the struts 158′ shown in FIGS. 33A-33C and described elsewhere herein.

Turning to FIG. 36C, yet another embodiment of a locator loop 250″ is shown that includes a collar 252,″ a plurality of struts 254,″ and an outer loop portion 256.″ Similar to the previous embodiments, the struts 254″ may include more flexible outer portions 254a″ and more rigid inner portions 254b.″ In this embodiment, the inner portions 254b″ have portions removed to increase their flexibility, which may desired in some applications. Thus, by several parameters may be adjusted to modify the rigidity of the struts 254″ in a desired manner, e.g., their width, thickness, internal openings, and the like.

Turning to FIG. 37, yet another embodiment of a locator loop 350 is shown that includes a portion formed from a tube 359, and a portion formed from one or more wires 355. As shown, the locator loop 350 includes a collar 352 and inner portions 354b of struts formed from the tube 359, similar to the previous embodiments. Outer portions 354a of the struts and the outer loop portions (not shown) may be formed from the one or more wires 355, e.g., similar to any of the wire loop embodiments described elsewhere herein. The ends of the wires 355 may be attached to the tube 359, for example, by weaving the wires 355 into one or more holes formed in the tube 359. In addition or alternatively, the wires 355 may be further secured to the tube 359 by welding, bonding, crimping, and the like.

Optionally, the wires 355 may be formed from drawn and filled tubes (“DFTs”), which may be a composite of a Nitinol outer tubular wire and a core of radiopaque material (e.g., gold, platinum, iridium, and the like). DFT wire may provide radiopacity without adding bulky elements to the locator loop.

There may be several advantages of providing the outer loop portions and/or outer portions 354a of the struts from a wire structure. For example, a wire may have a smoother, more uniform profile along its length, which may allow higher strength and/or minimal post-processing (i.e., electro-polishing, sandblasting, etc.). In addition, a wire may have a microstructure where the metal grains are oriented along the length of the wire. In contrast, cutting portions of the locator loop from a tube, e.g., the outer loop portions, the tube may be cut at angles that are not parallel to the grain structure, which may result in grain orientation that is irregular and/or may weaken the resulting locator loop. In addition, loops cut from a tube require the loops to be folded or bent, which may increase localized stresses, which may result in failure or other damage to the locator loop during use.

However, laser cut tubing may allow the collar to have a relatively small profile. Alternatively, a separate tube or other structure may be provided as a base to which the struts may be attached, but such a structure may be constructed less accurately, as compared to a laser cut collar. For example, in an alternative embodiment, a section of heat shrink tubing may be used to secured struts to the underlying catheter, although the heat shrink tubing may have less strength than a metal or other laser cut collar. Further, laser cut tubing may provide increased flexibility, i.e., allowing the various components, struts, collar, and loop portions, to be changed to meet desired mechanical and/or other performance criteria.

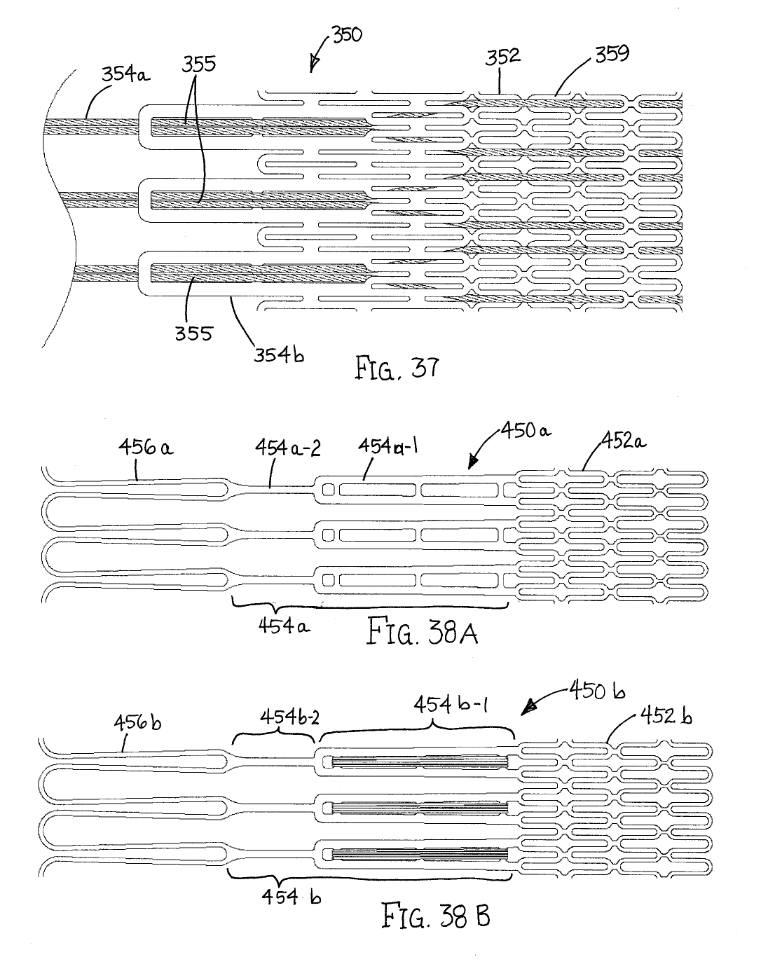

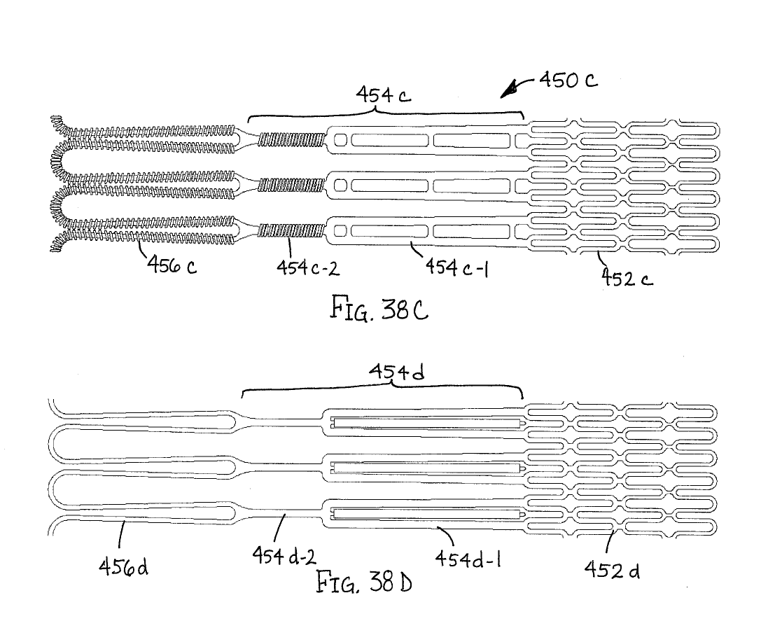

Turning to FIGS. 38A-38D, variations of these locator loops are shown, which may include components intended to enhance radiopacity of the locator loop. For example, as shown in FIG. 38B, radiopaque wire may be attached to inner portions 454b−1 of struts 454b of a locator loop 450b. Alternatively, as shown in FIG. 38C, radiopaque wire may be wrapped around the outer loop portions 456c of a locator loop 450c and/or around the outer portions 454c−2 of the struts 454c. In a further alternative shown in FIG. 38D, a radiopaque tube may be threaded or otherwise secured over a strut on inner support portions 454d−1 of the struts 454d of a locator loop 454d. These alternatives may be constructed and used similar to other embodiments described elsewhere herein. In addition, any of these embodiments for adding radiopacity may be included in any of the embodiments of locator loops or other structures described herein.

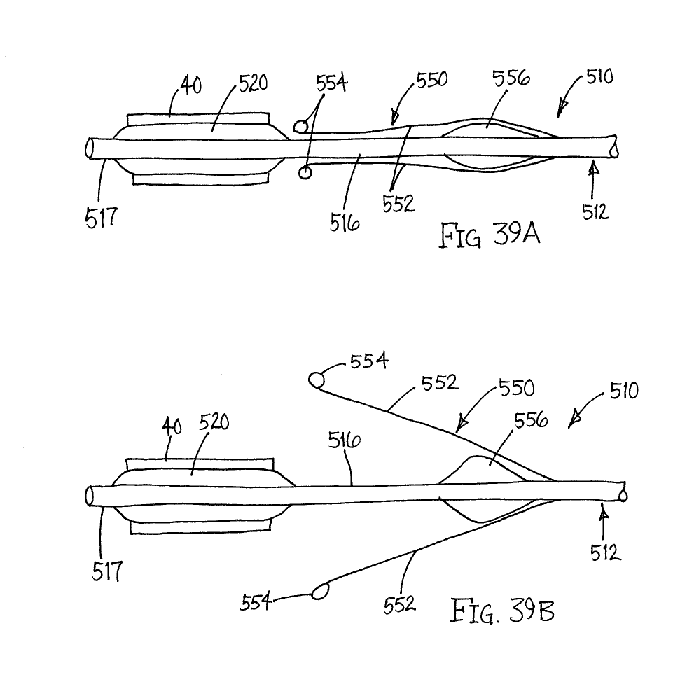

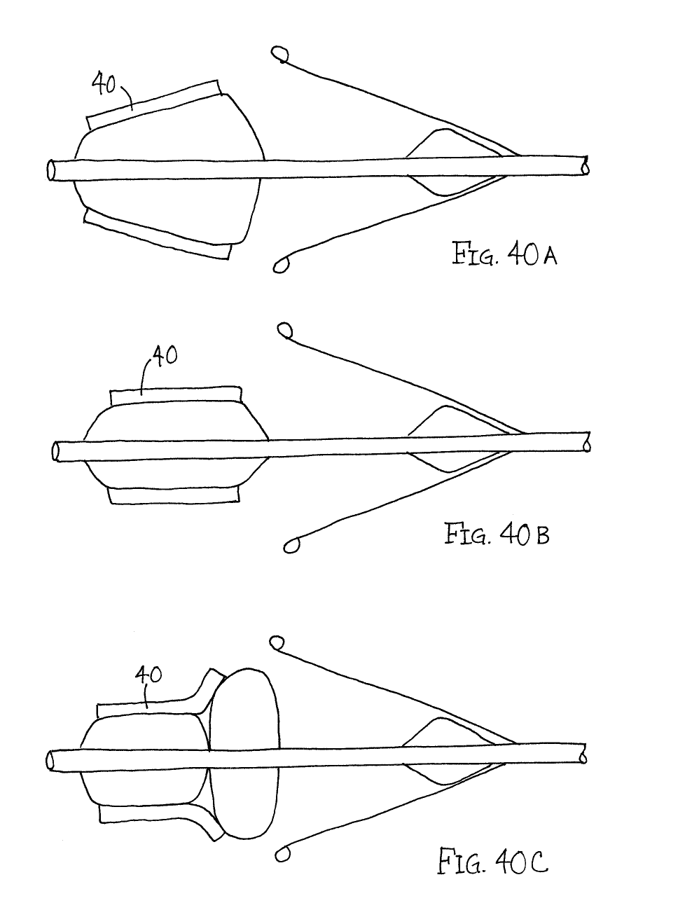

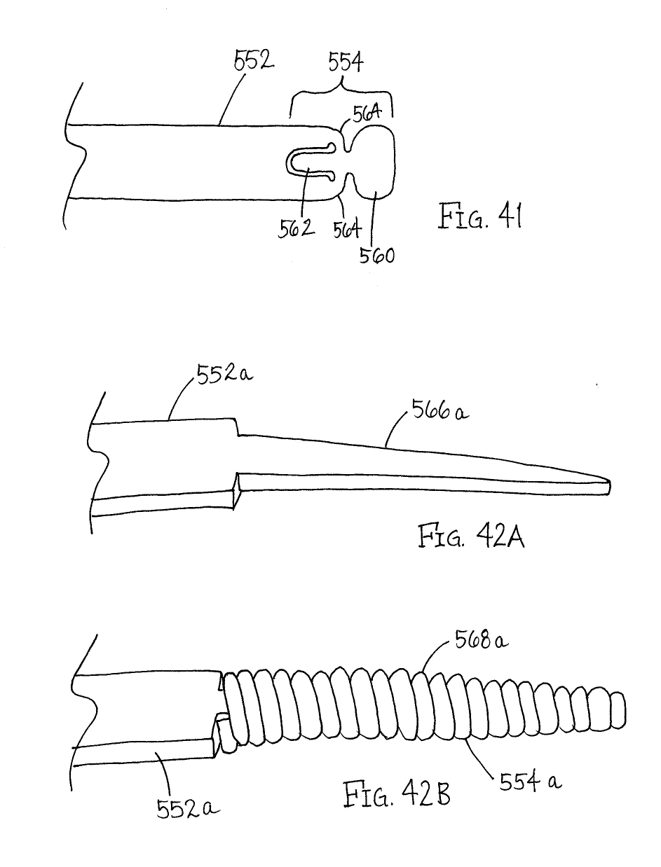

Turning to FIGS. 39A and 39B, another embodiment of an apparatus 510 is shown that includes a delivery catheter 512 including a distal end 516 carrying a stent 40 on a balloon 522, similar to previous embodiments. In addition, the apparatus 510 includes a locator device 550 including a pair of locator arms 552, each arm 552 including a fixed end attached to the distal end 516 of the delivery catheter 512 and a free end 554. In the embodiment shown, the locator arms 552 may be biased to an axial or contracted condition, such as that shown in FIG. 39A. The locator device 550 may include an actuator, e.g., a balloon 556 disposed on the distal end 516 of the delivery catheter 512. When the balloon 556 is inflated, the locator arms 552 may be deflected radially outwardly to an enlarged condition, such as that shown in FIG. 39B.

During use, the delivery catheter 512 may be introduced into a trunk adjacent a branch with the locator arms 552 in the contracted condition shown in FIG. 39A, e.g., similar to the methods described elsewhere herein. Within the trunk, the locator arms 552 may be expanded, as shown in FIG. 39B (e.g., after being advanced from a guide catheter, not shown), whereupon the distal end 516 of the delivery catheter 512 may be advanced into an ostium communicating with the branch. When the ends 554 of the locator arms 552 contact the wall surrounding the ostium, tactile feedback may be provided to the user, indicating that the stent 40 may be positioned within the ostium and/or branch. The stent may be deployed, similar to the other embodiments described herein, e.g., in a substantially uniform cylindrical configuration, such as that shown in FIG. 40B, in a tapered configuration, such as that shown in FIG. 40A, or in a flared configuration, such as that shown in FIG. 40C.

Once the stent 40 is expanded and/or otherwise deployed, the balloon 522 may be deflated, and the distal end 516 of the delivery catheter 512 withdrawn into a guide catheter or other sheath (not shown). The balloon 556 may be deflated, whereupon the locator arms 552 may resiliently resume the contracted condition, allowing the locator arms 552 to be withdrawn into the guide catheter. Alternatively or in addition, the locator arms 552 may be compressed towards the contracted condition when the distal end 516 of the delivery catheter 512 is withdrawn into the guide catheter, similar to the other embodiments described herein.

FIGS. 41-44B show various embodiments of substantially atraumatic tips that may be provided on the ends 554 of the locator arms 552 shown in FIGS. 39A and 39B, or in other embodiments of locator devices described herein.

For example, FIG. 41 shows a tip of a locator arm 552 including a free end 554 that includes a toe 560, a heel 562, and a pair of torsion bars 564. As the locator arm 552 makes contact with an object (e.g., a wall of a vessel, not shown), the toe 560 may hit first and cause a torque to be applied to the torsion bars 564. The torsion bars 564 may then bend in response to this load and the heel 562 and toe 560 may rotate relative to the rest of the locator arm 552. The rotation may continue until the heel 562 and toe 560 are parallel to the surface of the object making contact. The area of the heel 562, toe 560, and portions of the torsion bars 564 that contact the object may be relatively large relative compared to the to the force being applied, and therefore may be substantially atraumatic to the object being contacted.

FIGS. 42A and 42B show another exemplary embodiment of an atraumatic tip 554a that may be provided on a locator arm 552a. The locator arm 552a may be cut so that it has a tapered portion 566a on its free end. A coil 568a may then be placed over the tapered portion 566a, e.g., in a similar fashion as a guidewire tip, and may be soldered, welded, and/or bonded in place. Thus, the coil 568a may be provide an enlarged and/or resiliently deformable tip 554a, which may reduce the risk of perforation or other damage to a vessel wall contacted by the tip 554a.

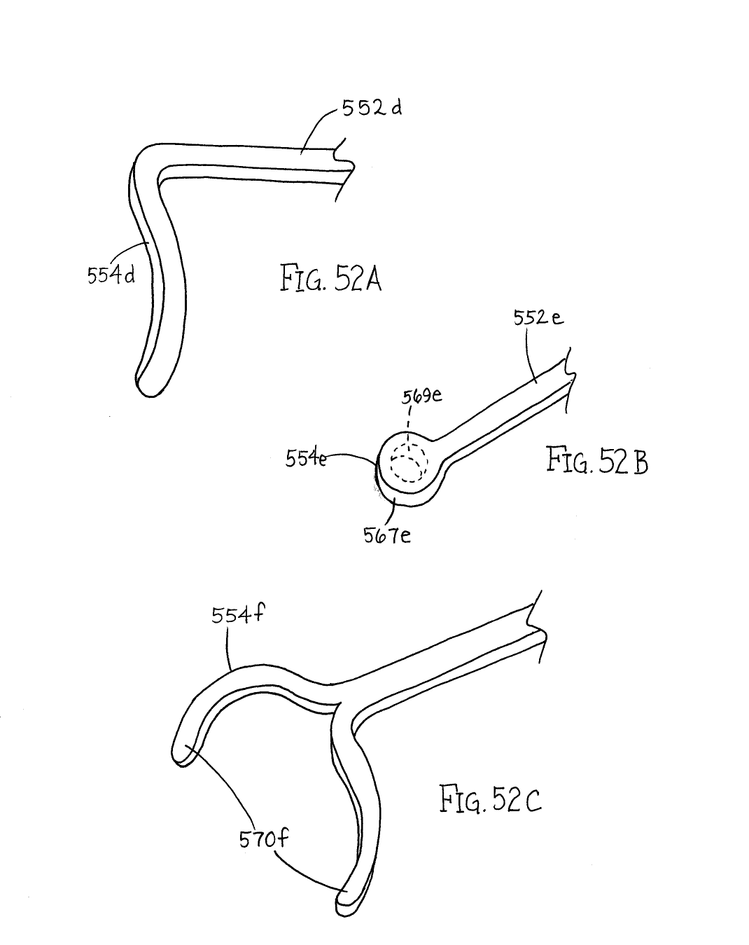

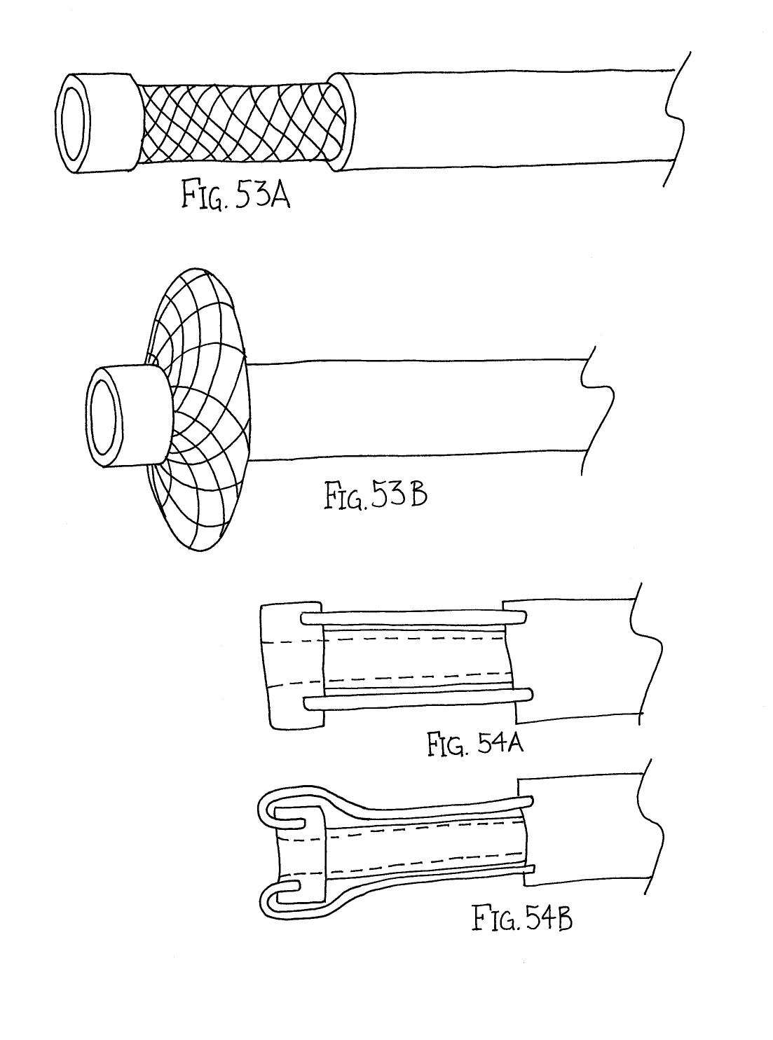

FIGS. 52A-52C show other alternative atraumatic tips that may be provided on locator arms described herein. For example, FIG. 52A shows an atraumatic tip 554d that may be provided on a locator arm 552d. The tip 554d may be formed by bending and/or curving the free end of the locator arm 552d, e.g., such that the tip 554d conforms substantially to the radius catheter (not shown) to which the locator arm 552d is attached. Thus, the tip 554d may extend around a portion of the circumference of the catheter while the locator arm 552d extends along a length of the catheter, which may minimize a profile of the locator arm 552d in the contracted condition.

Alternatively, as shown in FIG. 52B, an atraumatic tip 554e may be provided on a locator arm 552e that includes an enlarged tab 567e. Optionally, the tab 567e may include a hole or recess 569e (shown in phantom), which may be filled with a radiopaque material, if desired, to facilitate monitoring the locator arm 552e using fluoroscopy or other external imaging. In a further alternative, shown in FIG. 52C, an atraumatic tip 554f may be provided on a locator arm 552f that includes a pair of curved legs 570f. Similar to the atraumatic tip 554d described above, the legs 570f may include a radius corresponding to a radius of a catheter (not shown) to which the locator arm 552f is attached, e.g., to minimize a profile of the locator arm 552f in the contracted condition. Optionally, any of these features may be combined, e.g., provided together on a free end of a locator arm, such as including a tab on a curved leg (not shown).

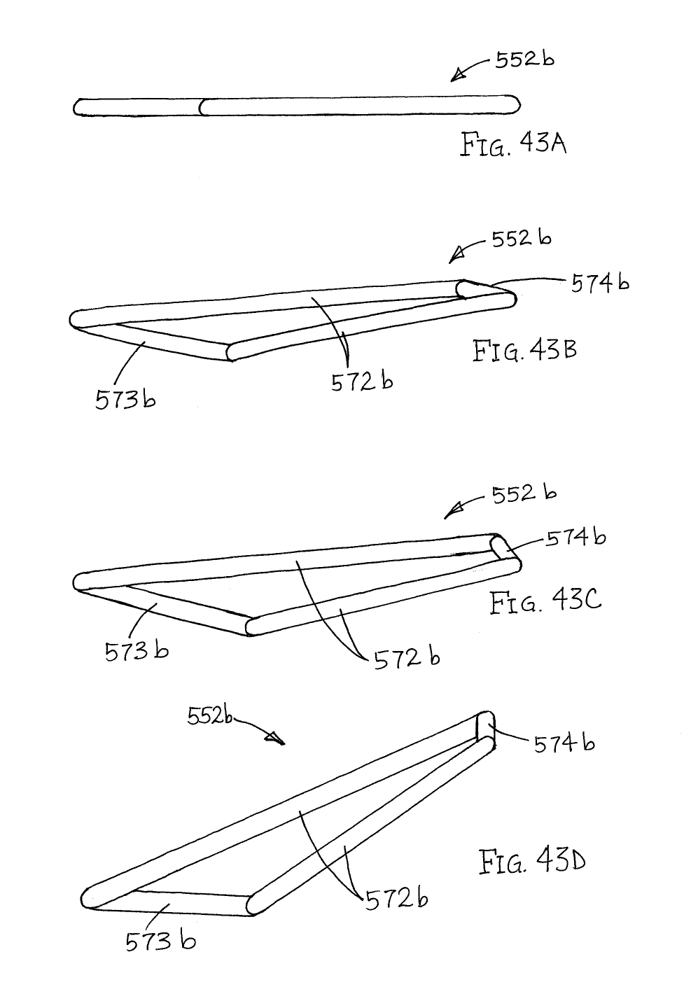

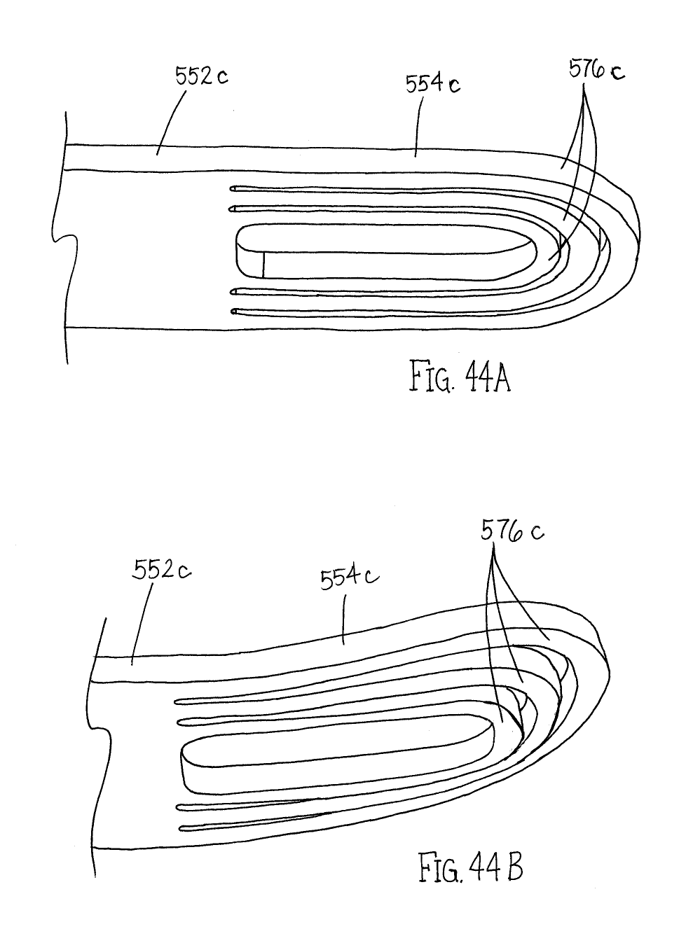

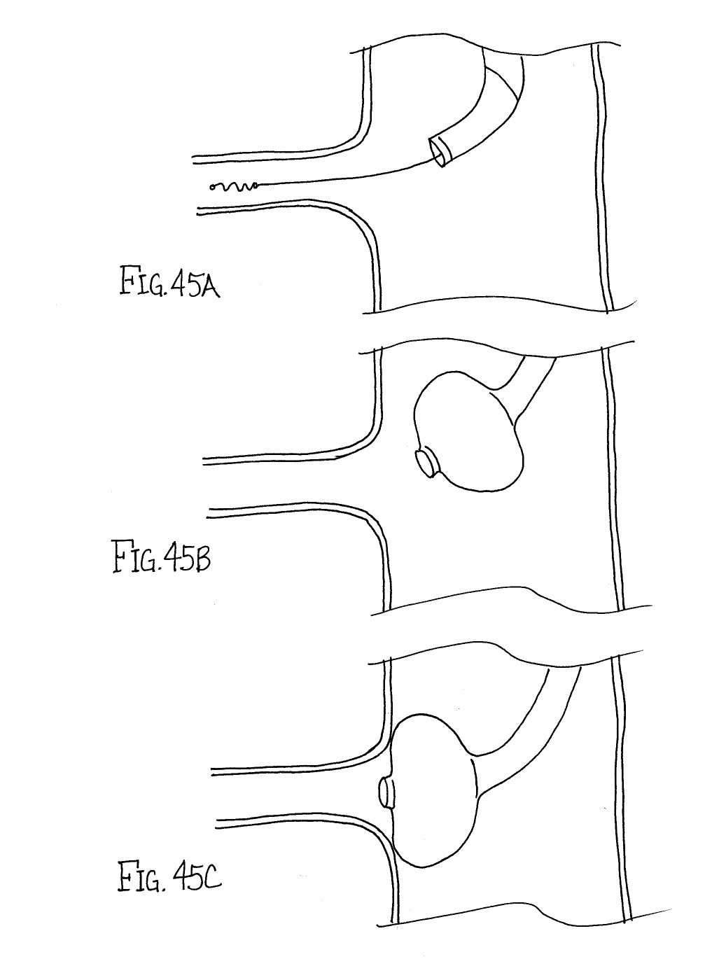

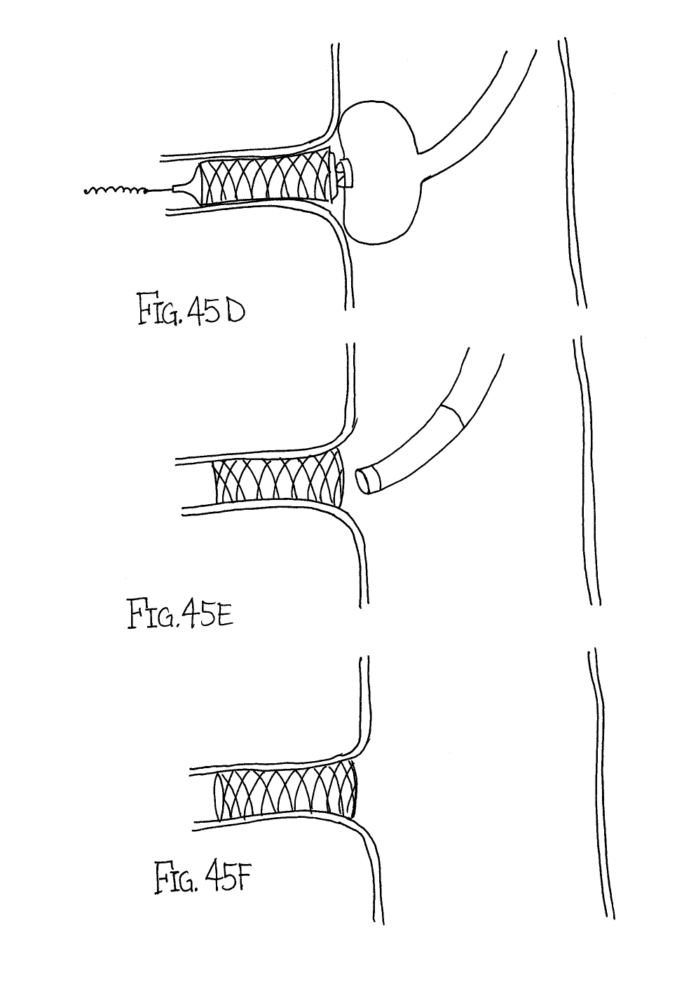

FIGS. 43A-43D show another embodiment of an atraumatic tip 554b for a locator arm 552b. In this set of Figures, the locator arm 552b may include three moveable members 572b, 574b and one static member 573b, which may be attached to a catheter or may simulate a surface on a delivery catheter (not shown) to which the members 572b are attached. As the members 572b are moved from their retracted state shown in FIG. 43A to a progressively more deployed state shown in FIGS. 43B-43D, the distal most member 574b may rotate into a vertical orientation. Because the vessel walls that will be contacted with the locator arm 552b are expected to be substantially perpendicular to the catheter shaft, the distal most locator arm 574b may be substantially parallel to that surface in the configuration shown in FIG. 43D, providing maximum surface area for atraumatic contact.

Turning to FIGS. 44A and 44B, an additional embodiment is shown for an atraumatic tip 554c for a locator arm 552c. As shown in FIG. 44B, a free end of the locator arm 552c may include a set of concentric loops 576c cut into the end of it. These loops 576c may be constructed, e.g., by thinning the loops 576c, so that the loops 576c are substantially flexible, e.g., may deform elastically or plastically when they come in contact with the patient's vasculature. FIG. 44B shows the atraumatic tip 554c being deflected after contacting a wall of a trunk surrounding an ostium (not shown). As can be seen, the loops 576c have deformed in response to the applied loads. This deformation prevents a large point load from being applied, and causing trauma to the patient.

FIGS. 45A-45F show an exemplary embodiment of a locator device provided on a guide catheter 1010 for locating and positioning the guide catheter 1010 relative to an ostium. Generally, the guide catheter may be constructed similar to the embodiments described elsewhere herein. Once properly positioned, the guide catheter may be used to deliver a stent 40 using a separate stent delivery catheter, such as those described in application Ser. No. 11/136,266, incorporated by reference above.

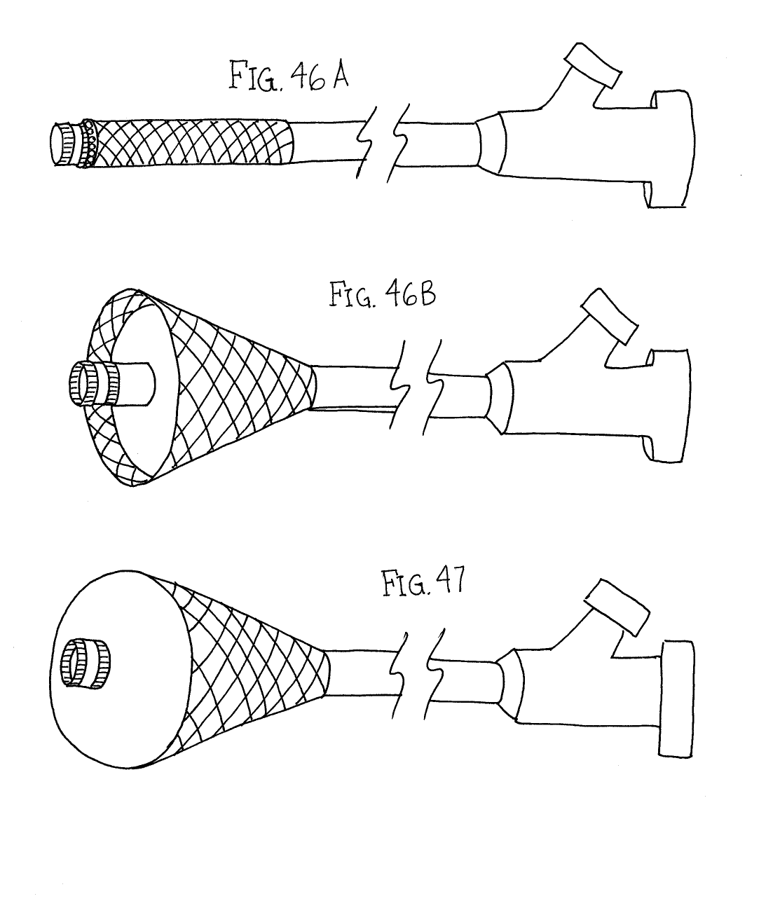

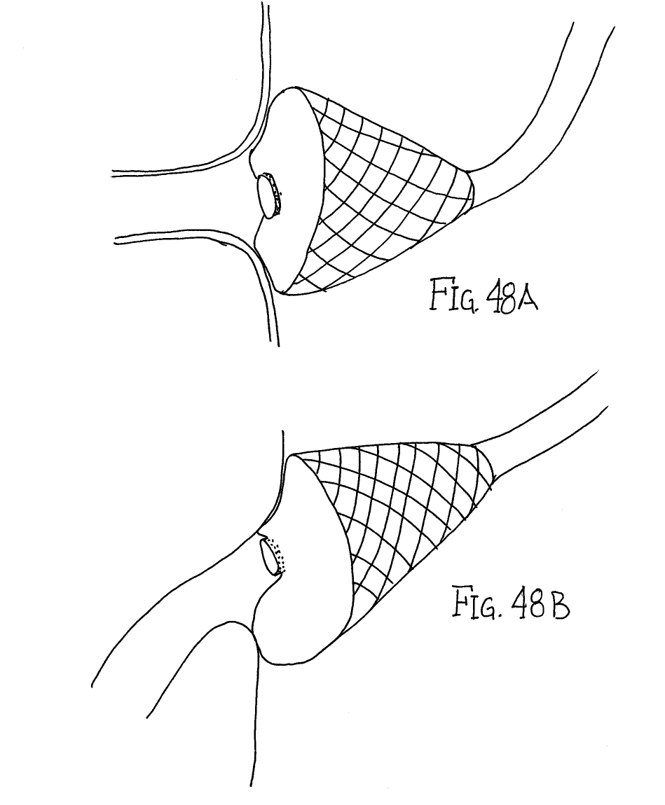

FIGS. 46A-48B show alternative embodiments of an expandable mesh or braid locator device that is expanded using an underlying balloon. FIGS. 50A and 50B show a plurality of splines or arms that may be expanded using an underlying balloon to provide a locator device. Any of these embodiments may be provided on a guide catheter or other tubular member. The guide catheter may then be used to locate and/or position the guide catheter adjacent an ostium, e.g., for delivering a stent into the ostium.

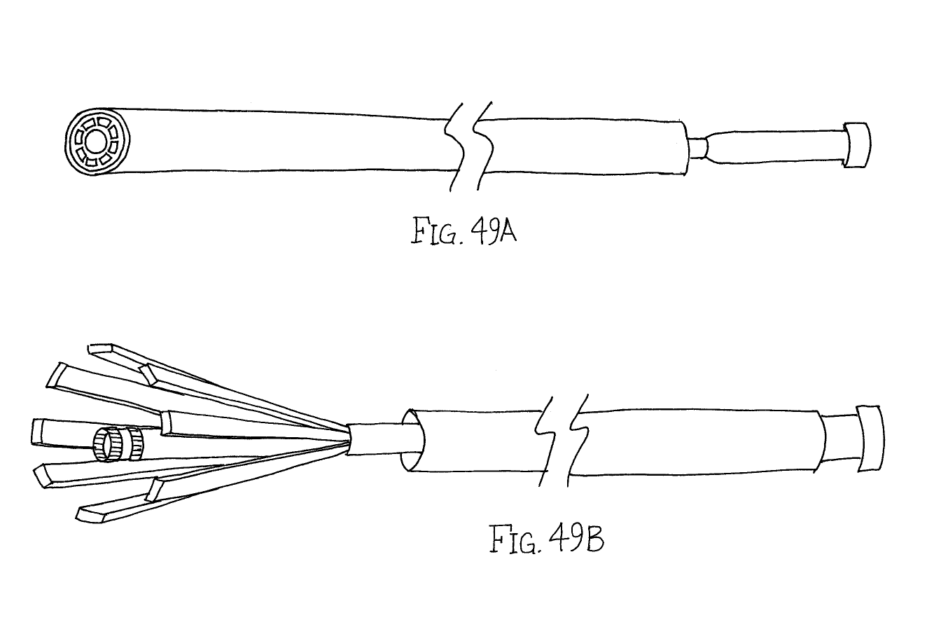

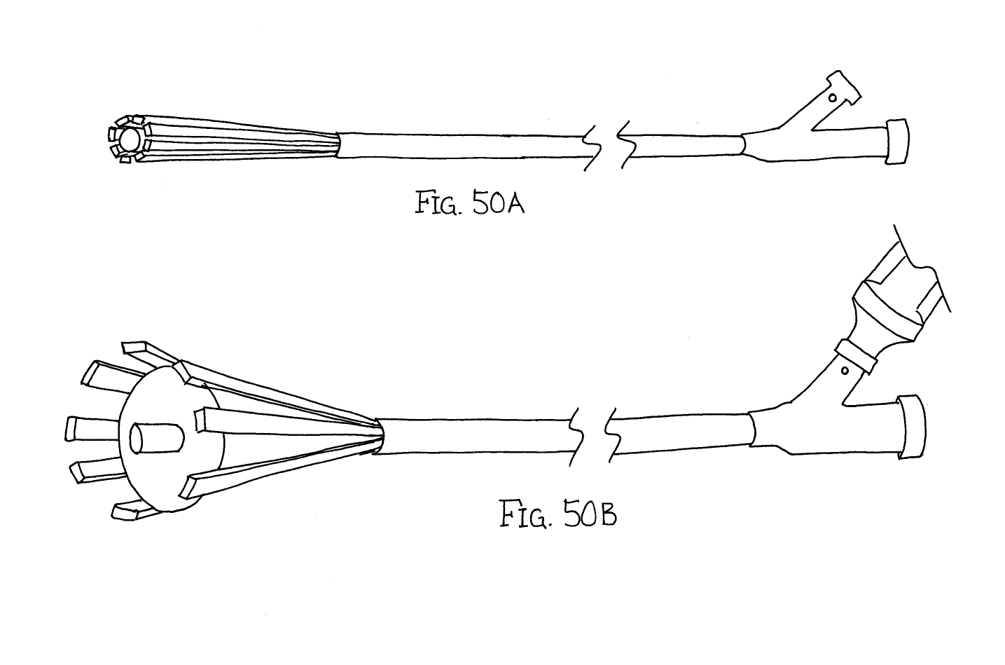

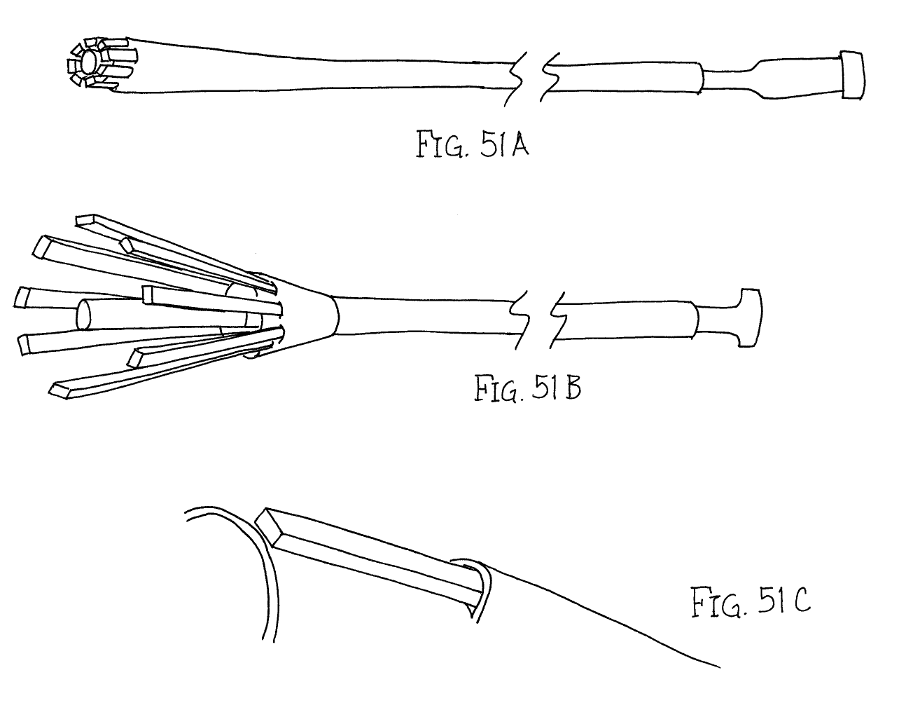

FIGS. 49A, 49B, 51A and 52B show embodiments of a locator device including a plurality of expandable arms or splines that are expanded by retracting an overlying sheath. The arms may be biased to extend outwardly when the sheath is retracted (FIGS. 49A and 49B) or may be actuated to expand using the sheath (FIGS. 51A and 51B).

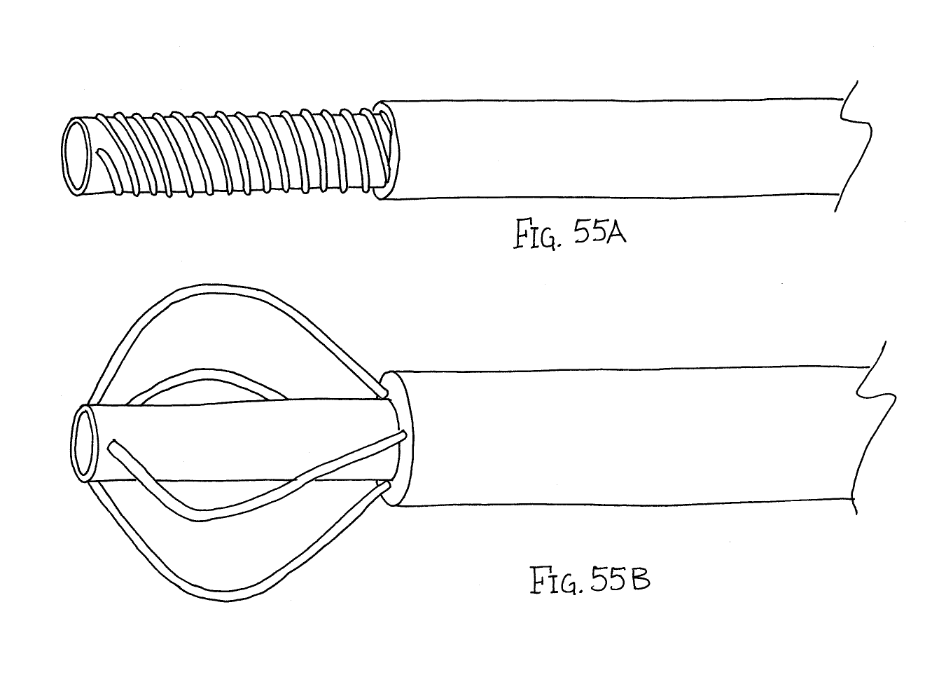

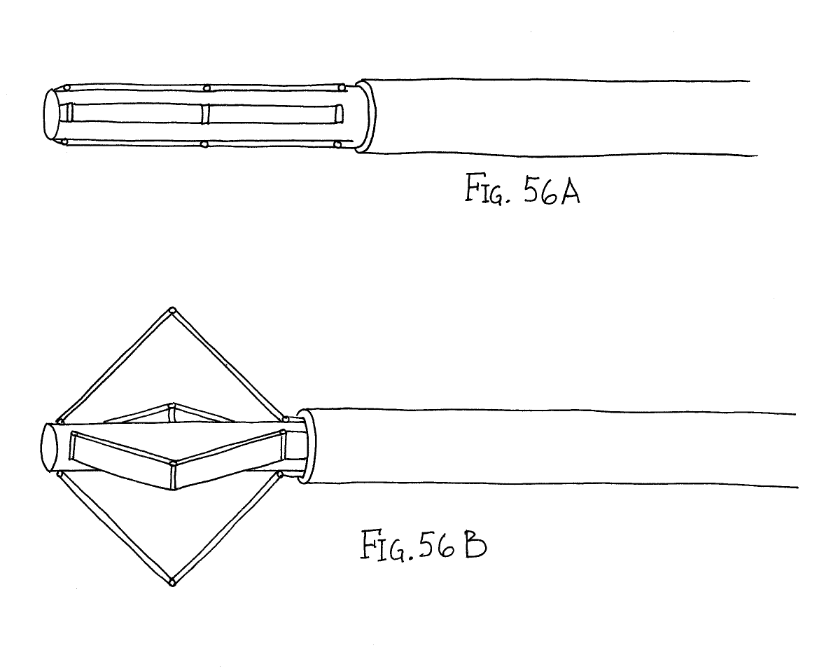

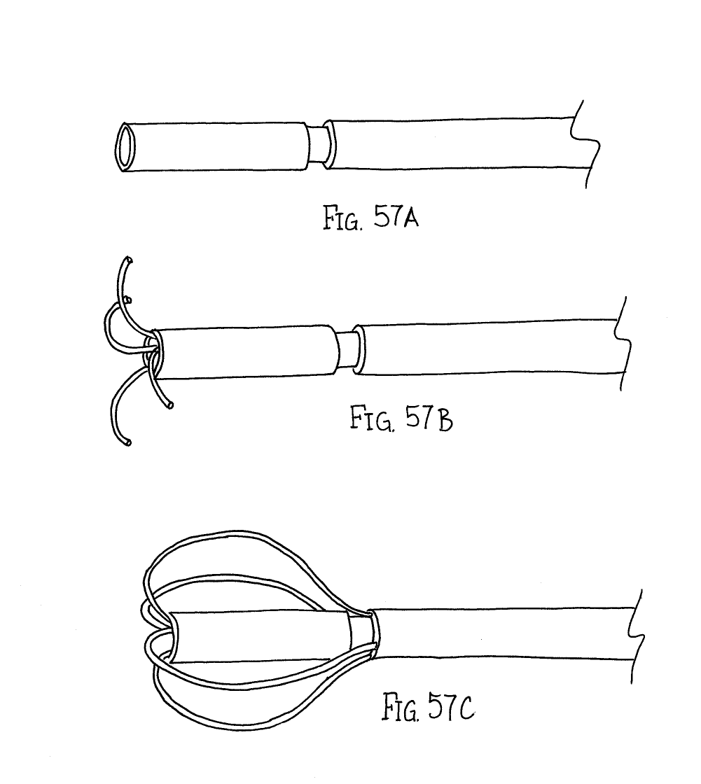

FIGS. 53A-54B show various embodiments of locator devices that include a braided mesh that may be compressed axially to cause the braid to buckle and expand radially outwardly. FIGS. 55A and 55B show a locator device including a plurality of splines or arms that may be unwound to expand and wound to contract. FIGS. 56A and 56B show a plurality of arms that may be preferentially buckled to expand radially outwardly to provide a locator device.

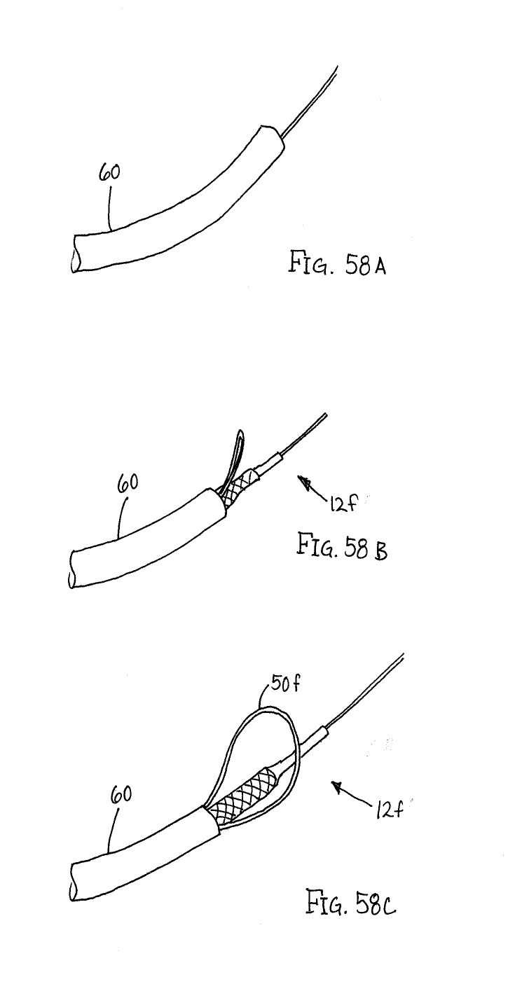

FIGS. 57A-57C show a plurality of wires or arms that may be deployed from a guide catheter. The arms may be biased to curve or evert back on themselves, e.g., until they engage a receiver on the guide catheter, thereby providing a locator device. It will be appreciated that nay of these locator devices may be provided on a stent delivery catheter, guide catheter, or other device that may be introduced into a patient's body, e.g., using the methods described elsewhere herein.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for locating an ostium of a body lumen, comprising:
a tubular member comprising a proximal end, a distal end sized for introduction into a body lumen, and a lumen extending between the proximal and distal ends;
an elongate member comprising a distal portion disposed within the lumen such that the distal portion may be advanced beyond the tubular member distal end; and
an expandable loop on the distal portion, the loop comprising first and second ends fixed to the distal portion, first and second resilient struts extending from the first and second ends, respectively, and a curved intermediate region extending between the first and second struts, the loop being resiliently compressible to a contracted condition when the distal portion is disposed within the lumen and resiliently expandable to an enlarged condition when the distal portion is advanced beyond the tubular member distal end.

2. The apparatus of claim 1, wherein the intermediate region defines an arcuate shape when the loop expands to the enlarged condition.

3. The apparatus of claim 2, wherein the arcuate shape defines a portion of an ellipse surrounding the distal portion.

4. The apparatus of claim 1, wherein the first and second struts extend transversely from the distal portion when the loop expands to the enlarged condition.

5. The apparatus of claim 4, wherein the first and second struts extend distally from the distal portion when the loop expands to the enlarged condition.

6. The apparatus of claim 5, further comprising a tubular prosthesis on the distal portion adjacent the first and second ends, the first and second struts extending over a portion of the prosthesis when the loops expands to the enlarged condition.

7. The apparatus of claim 1, wherein the loop substantially defines a plane in the enlarged condition, the plane defining an acute angle with a longitudinal axis of the tubular member.

8. The apparatus of claim 7, wherein the loop comprises an outer tip disposed away from the first and second ends, the outer tip extending out of the plane.

9. The apparatus of claim 1, wherein only a single loop is provided on the distal portion of the tubular member.

10. The apparatus of claim 1, wherein the expandable loop comprises a plurality of expandable loops.

11. The apparatus of claim 10, wherein the plurality of expandable loops are disposed asymmetrically around a circumference of the tubular member.

12. The apparatus of claim 11, wherein the plurality of expandable loops comprises curved intermediate regions that generally define a portion of an ellipse surrounding the distal portion.

13. The apparatus of claim 1, wherein the loop substantially defines a “D” shape in the enlarged condition.

14. The apparatus of claim 1, wherein the loop generally defines a curved shape in the enlarged condition define a surface extending transversely relative to a longitudinal axis of the tubular member.

15. The apparatus of claim 1, wherein the loop comprises a plurality of wires wound around one another and extending between the first and second ends.

16. The apparatus of claim 1, wherein the loop comprises a curved banana-peel shape in the enlarged condition.

17. The apparatus of claim 1, wherein the loop is twisted asymmetrically relative to a longitudinal axis of the tubular member in the enlarged condition.

18. The apparatus of claim 17, wherein the loop generally defines a plane in the enlarged condition, and wherein a normal axis extending from the plane does not extend substantially parallel to the longitudinal axis of the tubular member.

19. The apparatus of claim 17, wherein the first strut extends more axially than the second strut in the enlarged condition.

20. The apparatus of claim 17, wherein the first strut has a length that is shorter than the second strut.

21. The apparatus of claim 1, wherein the struts are resiliently deflectable when the loops expands to the enlarged condition to provide tactile feedback when the intermediate region contacts an ostium.

22. The apparatus of claim 1, wherein the struts extend axially when the loop is in the contracted condition.

23. The apparatus of claim 22, wherein the intermediate region defines a serpentine shape when the loop is in the contracted condition.

24. The apparatus of claim 1, wherein the loop comprises at least one of a wireform and a slotted tube.

25. An apparatus for locating an ostium of a body lumen, comprising:
a tubular member comprising a proximal end, a distal end sized for introduction into a body lumen, and a lumen extending between the proximal and distal ends;
an elongate member comprising a distal portion disposed within the lumen such that the distal portion may be advanced beyond the tubular member distal end; and
a plurality of expandable loops on the distal portion, each loop comprising first and second resilient struts extending from the distal portion, and a curved intermediate region extending between the first and second struts, each loop being resiliently compressible to a contracted condition when the distal portion is disposed within the lumen and resiliently expandable to an enlarged condition when the distal portion is advanced beyond the tubular member distal end, the loops being disposed around the distal portion such that the intermediate regions define at least a portion of an ellipse surrounding the distal portion when the loops are in the enlarged condition.

26. The apparatus of claim 25, wherein the first and second struts extend transversely from the distal portion when the loops expand to the enlarged condition.

27. The apparatus of claim 26, wherein the first and second struts extend distally from the distal portion when the loops expand to the enlarged condition.

28. The apparatus of claim 27, further comprising a tubular prosthesis on the distal portion adjacent the loops such that the first and second struts extend over a portion of the prosthesis when the loops expand to the enlarged condition.

29. The apparatus of claim 25, wherein the struts are resiliently deflectable when the loops are expanded to the enlarged condition to provide tactile feedback when one or more of the intermediate regions contact an ostium.

30. The apparatus of claim 25, wherein the struts extend axially when the loops are in the contracted condition.

31. The apparatus of claim 30, wherein the intermediate regions define a serpentine shape when the loops are in the contracted condition.

32. The apparatus of claim 25, wherein the loops comprise at least one of a wireform and a slotted tube.

33. An apparatus for locating an ostium of a body lumen, comprising:
a tubular member comprising a proximal end, a distal end sized for introduction into a body lumen, a lumen extending between the proximal and distal ends, and a distal portion;
one or more locator elements disposed asymmetrically on the distal portion, each locator element comprising a first end fixed to the distal portion and a second end free from the distal portion, each locator element being resiliently compressible to a contracted condition when the distal portion is disposed within a lumen of a delivery device, each locator element being resiliently expandable to an enlarged condition when fully deployed from the delivery device; and
a stent on the distal portion.

34. The apparatus of claim 33, wherein only a single locator element is disposed on the distal portion, the locator element extending transversely from the distal portion in the enlarged condition.

35. The apparatus of claim 33, wherein the one or more locator elements comprise a plurality of locator elements, the locator elements disposed adjacent one another around only a portion of a circumference of the tubular member.

36. The apparatus of claim 35, wherein the plurality of locator elements comprise outer curved portions that together define only a portion of an ellipse extending around a portion of the distal portion.

37. The apparatus of claim 33, wherein the one or more locator elements comprise one or more wire loops.

38. An apparatus for locating an ostium of a body lumen, comprising:
a tubular member comprising a proximal end, a distal end sized for introduction into a body lumen, a lumen extending between the proximal and distal ends, and a distal portion; and
a locator loop on the distal portion that is resiliently compressible to a contracted condition when the distal portion is disposed within a lumen of a delivery device and is resiliently expandable to an enlarged condition when fully deployed from the delivery device, the locator loop comprising a loop that substantially surrounds the distal portion of the tubular member in the enlarged condition, and a plurality of struts extending between the loop and the distal portion for attaching the locator loop to the tubular member.

39. The apparatus of claim 38, wherein the struts comprise an inner portion closer to the tubular member and an outer portion closer to the loop, the inner portion being more rigid than the outer portion.

40. The apparatus of claim 39, wherein the inner portion has at least one of a width and a thickness that is greater than the outer portion.

41. The apparatus of claim 38, further comprising one or more supports extending between adjacent struts at intermediate regions of the struts.

42. The apparatus of claim 38, wherein the locator loop comprises a base attached to the distal portion of the tubular member, the struts extending from the base.

43. The apparatus of claim 42, wherein the base and the struts are formed from a unitary tubular body.

44. The apparatus of claim 43, wherein the loop is formed from the unitary tubular body.

45. The apparatus of claim 43, wherein the loop defines a serpentine shape in the contracted condition, the loop being heat treated to expand resiliently to the enlarged condition from the serpentine shape when deployed from the delivery device.

46. The apparatus of claim 43, wherein the loop is formed from one or more wires attached to the unitary tubular body.

47. A method for delivering a stent within an ostium communicating from a main body lumen to a branch body lumen, comprising:
advancing a distal end of a delivery catheter into the main body lumen, the distal end comprising one or more locator elements constrained in a contracted condition;
releasing the one or more locator elements within the main body lumen, the one or more locator elements resiliently expanding asymmetrically;
directing the one or more locator elements against a wall of the ostium, the one or more locator elements causing the distal end of the delivery catheter to rotate about its longitudinal axis; and
performing a procedure at or within the ostium based upon the position of the one or more locator elements in the enlarged condition.

48. The method of claim 47, further comprising advancing the distal end after the one or more locator elements expand towards the enlarged condition, the one or more locator elements providing tactile feedback resisting further advancement when the one or more locator elements contact the main body lumen wall adjacent the ostium.

49. The method of claim 47, wherein the procedure comprises delivering a stent within at least one of the ostium and the branch.

50. A method for delivering a stent within an ostium communicating from a main body lumen to a branch body lumen, comprising:
advancing a distal end of a delivery catheter into the main body lumen, the distal end comprising one or more locator elements constrained in a contracted condition;
releasing the one or more locator elements within the main body lumen, the one or more locator elements resiliently expanding to substantially surround the distal end;
directing the one or more locator elements against a wall of the ostium, thereby causing one or more struts supporting the one or more locator elements to bend away from the ostium; and
performing a procedure at or within the ostium based upon the position of the one or more locator elements in the enlarged condition.
